(19) United States
(12) Patent Application Publication
(10) Pub. No.: US 2018/0325547 A1
(43) Pub. Date: Nov. 15, 2018

(54) Medical Tool for Reduced Force Penetration for Vascular Access

(71) Applicant: Actuated Medical, Inc., Bellefonte, PA (US)

(72) Inventors: Roger B. Bagwell, Bellefonte, PA (US); Douglas R. Dillon, Port Matilda, PA (US); Eric J. Hopkins, Bellefonte, PA (US); Olga M. Ocon-Grove, State College, PA (US); Brandon A. Pier, Altoona, PA (US); Eric M. Steffan, Karthaus, PA (US); Maureen L. Mulvihill, Bellefonte, PA (US); Casey A. Scruggs, Middleburg, PA (US); Ryan S. Clement, State College, PA (US)

(73) Assignee: Actuated Medical, Inc., Bellefonte, PA (US)

(21) Appl. No.: 16/029,251

(22) Filed: Jul. 6, 2018

Related U.S. Application Data
(63) Continuation-in-part of application No. 14/522,681, filed on Oct. 24, 2014.
Application No. 14/522,681 is a continuation-in-part of application No. 14/329,177, filed on Jul. 11, 2014, now abandoned.
Application No. 14/329,177 is a continuation of application No. 13/672,482, filed on Nov. 8, 2012, now Pat. No. 8,777,871.
Application No. 13/672,482 is a continuation of application No. 12/559,383, filed on Sep. 14, 2009, now Pat. No. 8,328,738.
Application No. 12/559,383 is a continuation-in-part of application No. 12/163,071, filed on Jun. 27, 2008, now Pat. No. 8,043,229.
(60) Provisional application No. 62/529,135, filed on Jul. 6, 2017.
(60) Provisional application No. 60/937,749, filed on Jun. 29, 2007.
(60) Provisional application No. 61/895,789, filed on Oct. 25, 2013.
(60) Provisional application No. 61/089,756, filed on Sep. 15, 2008.

(51) Int. Cl. A61B 17/34; A61M 5/20; A61B 5/00; A61M 5/158

(52) U.S. Cl. CPC A61B 17/3403; A61M 5/20; A61B 5/4896; A61M 5/158; A61B 2017/0011; A61B 17/3423; A61M 2205/582

(57) ABSTRACT

A device for penetrating tissue for fluid collection and delivery is provided having a driving actuator interconnected to and driving axial reciprocating motion of a penetrating member. A hollow member attached between the penetrating member and a reservoir permits axial reciprocation of the penetrating member while isolating the vibrations from the reservoir. A handpiece allows for one-handed use of the device. A slider device attached to the reservoir permits one-handed delivery and extraction of materials from the reservoir.

### CROSS-REFERENCE TO RELATED APPLICATIONS

This application claims the benefit of co-pending U.S. Provisional Application Ser. No. 62/529,135 filed on Jul. 6, 2017, which is incorporated by reference herein in its entirety for all purposes. This application is also a continuation-in-part of co-pending U.S. patent application Ser. No. 14/522,681 filed on Oct. 24, 2014, which is a continuation-in-part application of U.S. application Ser. No. 14/329,177, filed on Jul. 11, 2014, now abandoned, which is a continuation application of U.S. application Ser. No. 13/672,482, filed on Nov. 8, 2012, which issued as U.S. Pat. No. 8,777,871 on Jul. 15, 2014, which is a continuation application of U.S. application Ser. No. 12/559,383, filed on Sep. 14, 2009, which issued as U.S. Pat. No. 8,328,738 on Dec. 11, 2012, which is a continuation-in-part application of U.S. application Ser. No. 12/163,071 filed on Jun. 27, 2008, which issued as U.S. Pat. No. 8,043,229 on Oct. 25, 2011, which claims the benefit of U.S. Provisional Application Ser. No. 60/937,749 filed on Jun. 29, 2007, now expired, all of whose entire disclosures are incorporated by reference herein in their entireties for all purposes. U.S. patent application Ser. No. 14/522,681 also claims the benefit of U.S. Patent Application Ser. No. 61/895,789 filed on Oct. 25, 2013, now expired, which is incorporated by reference herein in its entirety for all purposes. U.S. patent application Ser. No. 12/559,383 also claims the benefit of U.S. Patent Application Ser. No. 61/089,756 filed on Sep. 15, 2008, now expired, which is incorporated by reference herein in its entirety for all purposes.

### STATEMENT REGARDING FEDERALLY SPONSORED RESEARCH OR DEVELOPMENT

This invention was made with government support under RR024943, AG037214, and OD023024 awarded by the National Institutes of Health, and 2013-33610-20821 awarded by the USDA. The government has certain rights in the invention.

### FIELD OF THE INVENTION

The present invention generally pertains to handheld medical, veterinary, and pre-clinical or laboratory research devices, and more specifically to electrically driven lancets, needles, epidural catheter inserters, biopsy instruments, vascular entry instruments, spinal access needles, and other catheterization needles. The invention is applicable to the delivery and removal of blood, tissues, medicine, nutrients, or other materials within the body.

### BACKGROUND

In the fields of medicine, veterinary, and pre-clinical or laboratory research the need to insert penetrating members (such as needles and lancets) into living tissues is ubiquitous. Some of the reasons necessitating tissue penetration and insertion of penetrating members include: to inject medications and vaccines, to obtain samples of bodily fluids such as blood, to acquire a tissue sample such as for biopsy, or to provide short or long term access to the vascular system such as intravenous (IV) catheter placement.

Of the 39 million patients hospitalized in the United States, 31 million (80%) receive an IV catheter for nutrition, medication, and fluids. Obtaining peripheral venous access is complicated by loose tissue, scar tissue from repeat sticks, hypotension, hypovolemic shock, and/or dehydration. These factors manifest in easily collapsed veins, rolling veins, scarred veins, and fragile veins making venipuncture problematic. Most hospitals allow a clinician to make several attempts at peripheral IV access before the hospital “IV team” is called. Studies have shown that success can improve significantly with experience. There are also a number of techniques that can be used such as tourniquets, nitroglycerin ointment, hand/arm warming, but these require additional time, are cumbersome, and do not work effectively in all situations. Tools are also available to improve visualization of the vasculature that use illumination, infrared imaging, or ultrasound. These tools, however, do not simplify peripheral venous access into a collapsible vein. In emergency situations, a clinician will often insert a central venous catheter (CVC) or possibly an intraosseous line. These procedures are more invasive, costly, and higher risk. Multiple needle sticks significantly increase patient anxiety and pain, leading to decreased patient cooperation, vasoconstriction, and greater opportunity for infection and complications. Repeated attempts to obtain venous access are costly to the healthcare facility; estimated at over $200,000 annually for a small hospital. In endoscopy facilities, which see large numbers of older patients, the problem is further exacerbated by fasting requirements that decreases the pressure in the veins. During cannulation, the needle and catheter push the near wall of the vein into the far wall, collapsing the vein—inhibiting the ability to place the needle into the inner lumen of the vein.

Tissue deformation during needle insertion is also an issue for soft tissue biopsy of tumors or lesions. Conventional needles tend to deform the tissue during the insertion, which can cause misalignment of the needle path and the target area to be sampled. The amount of tissue deformation can be partially reduced by increasing the needle insertion velocity, and so this property has been exploited by biopsy guns on the market today.

Blood sampling is one of the more common procedures in biomedical research involving laboratory animals, such as mice and rats. A number of techniques and routes for obtaining blood samples exist. Some routes require/recommend anesthesia (such as jugular or retro-orbital), while others do not (such as tall vein/artery, saphenous vein or submandibular vein). All techniques utilize a sharp (lancet, hypodermic needle, or pointed scalpel) that is manually forced into the tissue to produce a puncture that bleeds. A capillary tube is positioned over the puncture site to collect the blood droplets for analysis, or the blood may be collected into a syringe or vacuum vial. Regardless of the sharp used, if an individual is properly trained the procedure can be performed quickly to minimize pain and stress. It is important to minimize stress as this can interfere with blood chemistry analysis, particularly for stress-related hormones. Another much more expensive strategy is to place an indwelling catheter and obtain blood samples in an automated device. However, the catheter cannot be left in over the life span. In addition, the tethering jackets and cables, which must remain in contact with the animal, will likely cause stress. Microneedles can be implanted with highly reduced insertion force and less pain, but may not produce a large enough puncture to yield significant blood for collection and analysis.

Research supports that needle vibration, or oscillation, causes a reduction in needle insertion forces. The increased needle velocity from oscillation results in decreased tissue deformation, energy absorbed, penetration force, and tissue damage. These effects are partly due to the viscoelastic properties of the biological tissue and can be understood through a modified non-linear Kelvin model that captures the force-deformation response of soft tissue. Since internal tissue deformation for viscoelastic bodies is dependent on velocity, increasing the needle insertion speed results in less tissue deformation. The reduced tissue deformation prior to crack extension increases the rate at which energy is released from the crack, and ultimately reduces the force of rupture. The reduction in force and tissue deformation from the increased rate of needle insertion is especially significant in tissues with high water content such as soft tissue. In addition to reducing the forces associated with cutting into tissue, research has also shown that needle oscillation during insertion reduces the frictional forces between the needle and surrounding tissues.

Recently, a number of vibration devices have been marketed that make use of the Gate's Control Theory of Pain. The basic idea is that the neural processing, and therefore perception of pain, can be minimized or eliminated by competing tactile sensations near the area of pain (or potential pain) originates. Vibrational devices may be placed on the skin in attempt to provide “vibrational anesthesia” to an area prior to, or possibly during, a needle insertion event. Research has shown that tissue penetration with lower insertion forces results in reduced pain. The Gate Control Theory of Pain provides theoretical support for the anesthetic effect of vibration. The needle vibration may stimulate non-nociceptive Aβ fibers and inhibit perception of pain and alleviate the sensation of pain at the spinal cord level. In nature, a mosquito vibrates its proboscis at a frequency of 17-400 Hz to reduce pain and improve tissue penetration.

Other vibrating devices directly attach to a needle-carrying syringe and employ non-directional vibration of the needle during insertion. Reports suggest that this type of approach can ease the pain of needle insertion for administering local anesthetic during dental procedures, and to enhance the treatment of patients undergoing sclerotherapy. These non-directed vibration techniques do not allow for precise direct control of the needle tip displacements, and by their nature induce vibrations out of the plane of insertion, which could increase the risk for tissue damage during insertion. It would therefore be beneficial to have a device that vibrates a needle also attached to a fluid reservoir, such as a syringe, for direct and immediate fluid collection or delivery, but which could employ directional vibration for more precise control of the needle tip. Such a device should also be handheld for ease of use. Furthermore, existing vibrational devices for improving needle insertion cannot be readily integrated into a control system which would allow for the ability to control and/or maintain the magnitude of needle oscillation during insertion through a wide range of tissue types.

A need therefore exists to improve the insertion of penetrating members (such as needles, lancets, and syringes), by reducing the force required to insert them, causing less tissue deformation, and inducing less pain and stress to the patient, research subject, and clinician/researcher, even for collecting and delivering larger volumes of fluids, such as greater than 1 mL. As such, there remains room for variation and improvement within the art.

### SUMMARY

Various features and advantages of the invention will be set forth in part in the following description, or may be obvious from the description, or may be learned from practice of the invention.

The invention provides in one exemplary embodiment a handheld device that provides axially-directed oscillatory motion (also referred to as reciprocating motion) to a detachable penetrating member (such as but not limited to lancets, needles, epidural catheters, biopsy instruments, and vascular entry instruments) at a distal end, for use in procedures (such as but not limited to vascular entry, catheterization, and blood collection). The device comprises at least one linear reciprocating actuator that can be reversibly attached to a penetrating member or other composite system which itself contains a penetrating member, and wherein the driving actuator provides motion to the penetrating member, causing it to reciprocate at small or micro-level displacements, thereby reducing the force required to penetrate through tissues. Reciprocating motion of the penetrating member facilitates less tissue displacement and drag, enabling, for example, easier access into rolling or collapsed vasculature. Specific applications of the invention include, but are not limited to, penetration of tissues for delivery or removal of bodily fluids, tissues, nutrients, medicines, therapies, and placement or removal of catheters. This device is for inserting penetrating members into the body, including human or animal subjects, with or without an attached fluid reservoir, for a variety of applications including but not limited to blood sample collection and medication delivery.

The handheld device disclosed may be a driving actuator composed of a handpiece body housing at least one oscillatory linear actuator. The actuator is preferably a voice coil motor (VCM) but may alternatively be implemented with a DC motor, solenoid, piezoelectric actuator, or linear vibration motor disposed within the handpiece body. The driving actuator may be coaxial with, parallel to, perpendicular to, or at an oblique angle relative to the penetrating member. The actuator may cause a motor shaft to oscillate or vibrate back and forth relative to the handpiece body, which may be in the axial direction of the shaft. In certain embodiments, the actuator may cause the motor shaft to rotate in a rotational direction. Attached to one end of the shaft is a coupling mechanism, such as a motor linkage, which enables reversible attachment of a penetrating member (or to a separate device that already has a penetrating member attached to it).

The need for reversible attachment to a range of penetrating members or separate devices that employ a penetrating member, requires a number of different attachment schemes in order to cause linear, reciprocating motion of the penetrating member. In the preferred embodiment the handheld device has a coupler that enables reversible attachment of LUER-slip® (slip tip) or LUER-Lok® (LUER-Lock) style needle or lancet hubs. In another embodiment of the device, a custom connection enables reversible attachment of separate devices with a penetrating member (such as syringe with attached needle or a safety IV-access device) which allows the linear actuator to vibrate the composite system, thereby resulting in reciprocating motion being delivered to the attached penetrating member.

Additional features include embodiments that enable delivery or removal of fluids down the lumen of hollow penetrating members, such as but not limited to via side port that allows access to the inner lumen. Tubing that is sufficiently compliant so as not to impede the reciprocating motion of the actuator and penetrating member, is then used to channel fluid from a source or reservoir, such as a syringe, into the lumen for delivery of medication or other treatments. The side port which accesses the inner lumen of the penetrating member may also enable bodily fluids or tissues to be extracted by applying suction. In certain embodiments, the compliant tubing is coaxial with the penetrating member and the reservoir, such as a syringe, and permits transfer of fluid between the penetrating member and reservoir such as for blood sample collection or delivery of medications. In such embodiments, the tubing does not impede the reciprocating motion of the actuator and penetrating member, but isolates the vibrations of the penetrating member from the reservoir. This allows for smaller, more compact driving actuators to be used to obtain effective reduction of force from reciprocating oscillations of the penetrating member while minimizing vibrations throughout the rest of the device.

In some embodiments, however, vibration of the syringe may be desired. In such cases, other additional features include embodiments that enable delivery or removal of fluids through a side mounted syringe that oscillates back and forth relative to the handpiece body where the driving actuator is coupled to the syringe and supplies the oscillation or vibration to the syringe. A coupling mechanism is attached to the syringe that enables reversible or removable attachment of a penetrating member (or to a separate device that already has a penetrating member attached to it). This embodiment indudes a means to easily accomplish movement of the syringe plunger to a forward or backward position for delivery or removal of bodily fluids, tissues, nutrients, medicines, or therapies.

With regard to driving actuators in the handpiece that exhibit resonant behavior, such as the VCM actuator (discussed in embodiments presented below), the invention includes a set of methods by which to optimally operate the device in order to achieve desired oscillation amplitudes throughout the insertion of a penetrating member into target tissues. The resonant peak in the displacement versus frequency response of the driving actuator is influenced greatly by the loading from the tissue that interacts with the penetrating member. The reason for the change in the frequency response is because the penetrating member experiences frictional, inertial, and elastic forces that interact with the driving actuator, and the overall system exhibits an altered frequency response. By operating the device at some frequency above the resonant frequency of the driving actuator in air (for example >⅓ octave, but more optimally near ½ octave), the reciprocating motion can be maintained with very little, if any, damping for penetration of many tissue types.

Alternatively, a feedback loop can be constructed by employing a displacement sensor (such as, but not limited to, a linear variable differential transformer (LVDT) to continually monitor displacement and a controller that can continually adjust the operating frequency to keep it near the actual resonance frequency of the coupled system (tissue and driving actuator, coupled via penetrating member). By attempting to keep the operating frequency near resonance of the coupled system, power requirements of the device are greatly reduced. Keeping the system at resonance also mitigates the need to ‘overdrive’ the system, i.e., drive at a displacement or frequency greater than needed initially, which can contribute to unnecessary heating. The monitoring of the frequency and displacement of the system can also be used to signal the transducer to stop vibration when penetration of the desired tissue is complete.

Another feedback-based method of maintaining near constant oscillatory displacement amplitude during insertion of the penetrating member into variety of tissues, utilizes current control. With this method, the current amplitude supplied to the driving actuator is increased to overcome the damping effects of tissue on the reciprocating penetration member. Again, a displacement sensor can be employed to continually monitor displacement and adjust current amplitude to achieve the target displacement magnitude. Additional methods may deploy a combination of frequency and current control methods by which to maintain displacement. Other methods may not employ feedback but simply anticipate the loading effect of the target tissue and set the operating frequency or current such that optimal displacement amplitude is achieved at some point during the course of tissue penetration. The system may be off resonance when no load is encountered by the penetrating member. However, when the penetrating member penetrates tissue the loading causes the resonance of the system to move closer to the driving frequency such that no adjustments to the driving actuator are needed. In some instances the resonance of the system may be at the driving frequency in the loaded condition. In other arrangements, the driving actuator may be adjusted so that it is on resonance when in a loaded state, and is off resonance during no load conditions. In yet other arrangements, the operating frequency is not at a resonance frequency when in the no load condition, but the operating frequency is closer to the resonance frequency, as compared to the no load resonance frequency, when in the load condition.

The handheld device of the present invention may require an electrical power signal to excite an internal actuator. Upon excitation by the electrical signal, the driving actuator converts the signal into mechanical energy that results in oscillating motion of the penetrating member, such as an attached needle, lancet, epidural catheter, biopsy instrument, or vascular entry instrument.

Additionally, the invention with specific control electronics will provide reduction of force as the penetrating member is inserted and/or retracted from the body.

The device may also include a slider device that selectively or removably attaches to the reservoir and facilitates the easy operation of the reservoir for collection and delivery of fluids and materials therefrom. A guide shaft may extend along the reservoir, such as a syringe, and have a guide shaft coupling that removably connects to the plunger which is slidably inserted in the syringe. The guide shaft may also be slidably connected to the syringe body, such as through an adapter, so that when force is applied to the guide shaft in a distal or proximal direction, the guide shaft slides along the syringe body. In certain embodiments, the guide shaft coupling and the adapter may have the same geometry such that the slider device is reversible and may be attached to the reservoir in any direction. The guide shaft and plunger move together through the guide shaft coupling connection independent of the linear/axial reciprocations of the penetrating member that result from the driving actuator. The slider device may be separately attached to and removed from the device as desired.

These and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended Figs. in which:

FIG. 1A is a cross-sectional view of the preferred embodiment of the driving actuator handpiece utilizing a reciprocating VCM and LVDT sensor;

FIG. 1B is a cross-sectional view that illustrates the magnet assembly of the driving actuator (VCM);

FIG. 1C is a cross-sectional view that illustrates the VCM of FIG. 1A;

FIG. 2A is a side view of the driving actuator handpiece with a LUER-hub style penetrating member attached;

FIG. 2B is a close up view of the LUER-hub style penetrating member coupled to the distal tip of driving actuator handpiece;

FIG. 3A is a perspective view of the keyed coupler at the distal end of driving actuator handpiece which restricts rotational movement of the attached penetrating member;

FIG. 3B is a complete side view of the LUER compatible keyed coupler showing the space (keyway) allowed around the tabs (keys) of the coupler;

FIG. 3C is a perspective view of the keyed coupler and a rotating keyway head at the distal end of the driving actuator handpiece which provides controlled rotational movement while still allowing axial motion of the attached penetrating member;

FIG. 3D is a complete side view of the LUER compatible keyed coupler showing the space (keyway) allowed around the tabs (keys) of the coupler within the rotating keyway head;

FIG. 4 is a top plane view of the driving actuator handpiece with a mounted syringe connected to the side port of the LUER-hub of penetrating member for removal or injection of fluids;

FIG. 5 is a perspective view of the driving actuator handpiece with an incorporated foot switch for initiating and terminating power to the driving actuator;

FIG. 6A is a view of an embodiment of the driving actuator handpiece with an inline coupling sled attachment dipped to a safety IV device for the purpose of providing reciprocating motion to penetrating member;

FIG. 6B shows an isolated view demonstrating safety IV device attachment to coupling sled (driving actuator handpiece not shown);

FIG. 6C is a perspective view of the safety IV device after attached to the coupling sled;

FIG. 6D is a cross-sectional view that illustrates the driving actuator handpiece utilizing a reciprocating VCM that incorporates a coupling sled attachment clipped to a safety IV device;

FIG. 7A is a perspective view of an embodiment of the driving actuator handpiece with a side mounted syringe that is attached to the driving actuator to provide axially-directed oscillatory motion to the syringe and coupled penetrating member;

FIG. 7B is a side view of the embodiment of FIG. 7A that shows the guide shaft and coupled plunger in a forward position;

FIG. 7C is a side view of an embodiment of FIG. 7A that shows the guide shaft and coupled syringe plunger in a backward position;

FIG. 8A is a side view of an embodiment utilizing a geared slider for movement of the coupled syringe plunger and located in a forward position;

FIG. 8B is a side view of an embodiment utilizing a geared slider for movement of the coupled syringe plunger and located in a back position;

FIG. 8C is a cross-sectional view of an embodiment of FIG. 8A and FIG. 8B utilizing a geared slider to move the coupled syringe plunger forward and back;

FIG. 8D is a cross-sectional view of an alternate embodiment utilizing a double geared slider to move the coupled syringe plunger forward and back;

FIG. 9 is a graph showing typical displacement versus frequency behavior for VCM driving actuator in loaded and unloaded conditions;

FIG. 10A is a graphic demonstration of frequency-based displacement control method for overcoming the damping effect of tissue during a tissue penetration event using the driving actuator;

FIG. 10B is a graphic demonstration of a current-based control method for overcoming damping effect of tissue during a tissue penetration event using the driving actuator;

FIG. 11 is a graphic containing plots of displacement (oscillation amplitude) during the course of insertion of a penetrating member into tissue with driving actuator set to provide different displacement frequency and amplitude levels;

FIG. 12 is a graphical summary of insertion tests of a reciprocated 18G hypodermic needle into porcine skin with the driving actuator delivering different displacement frequency and amplitude levels;

FIG. 13 is a block diagram of electronics layout for voltage and current sensing applications.

FIG. 14 is an isometric view of another embodiment of the invention showing an oscillating needle insertion device.

FIG. 15 is an isometric view showing a first embodiment of a driving actuator and motor linkage in the oscillating needle insertion device of FIG. 14.

FIG. 16 is an isometric view of the motor linkage of FIG. 15.

FIG. 17 is an isometric view of FIG. 16 from an opposite perspective.

FIG. 18 is an isometric view of a second embodiment of an oscillating needle insertion device.

FIG. 19 is a partial isometric view of the driving actuator and motor linkage of the oscillating needle insertion device of FIG. 18.

FIG. 20 is a partial isometric view of a third embodiment of an oscillating needle insertion device.

FIG. 21 is a perspective view of a coupler of the oscillating needle insertion device of FIG. 20.

FIG. 22 is a perspective view of a motor linkage of the oscillating needle insertion device of FIG. 20.

FIG. 23 is a partial isometric view of a fourth embodiment of an oscillating needle insertion device utilizing a piezoelectric transducer.

FIG. 24 is an exploded view of the oscillating needle insertion device of FIG. 23.

FIG. 25 is a partial isometric view of the oscillating needle insertion device showing a coupling bracket to a fluid reservoir.

FIG. 26 is a partial isometric view of the oscillating needle insertion device showing a second embodiment of a coupling bracket to a fluid reservoir.

FIG. 27 is an exploded view of the penetrating member, hollow member and fluid reservoir of the oscillating needle insertion device.

FIG. 28 is an exploded view of the hollow member of FIG. 27.

FIG. 29 is a cross-sectional view of the hollow member of FIG. 27.

FIG. 30 is an isometric view of one embodiment of sliding device used with the oscillating needle insertion device, shown in a forward position.

FIG. 31 is an isometric view of the sliding device of FIG. 30, shown in a retracted position.

FIG. 32 is an exploded view of a second embodiment of the sliding device and a syringe reservoir.

Repeat use of reference characters in the present specification and drawings is intended to represent the same or analogous features or elements of the invention.

### DETAILED DESCRIPTION

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations.

It is to be understood that the ranges mentioned herein include all ranges located within the prescribed range. As such, all ranges mentioned herein Include all sub-ranges included in the mentioned ranges. For instance, a range from 100-200 also includes ranges from 110-150, 170-190, and 153-162. Further, all limits mentioned herein include all other limits included in the mentioned limits. For instance, a limit of up to 7 also includes a limit of up to 5, up to 3, and up to 4.5.

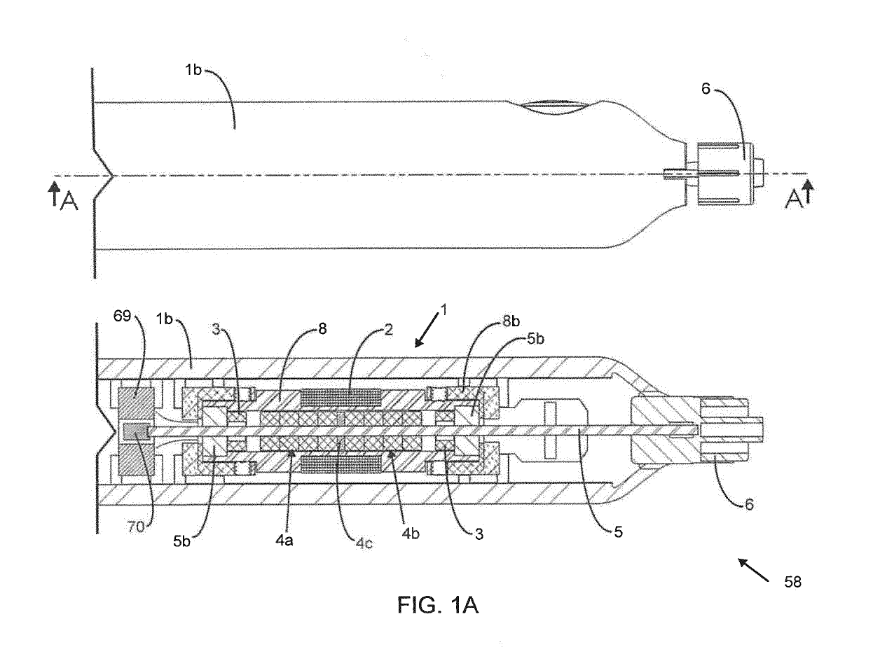

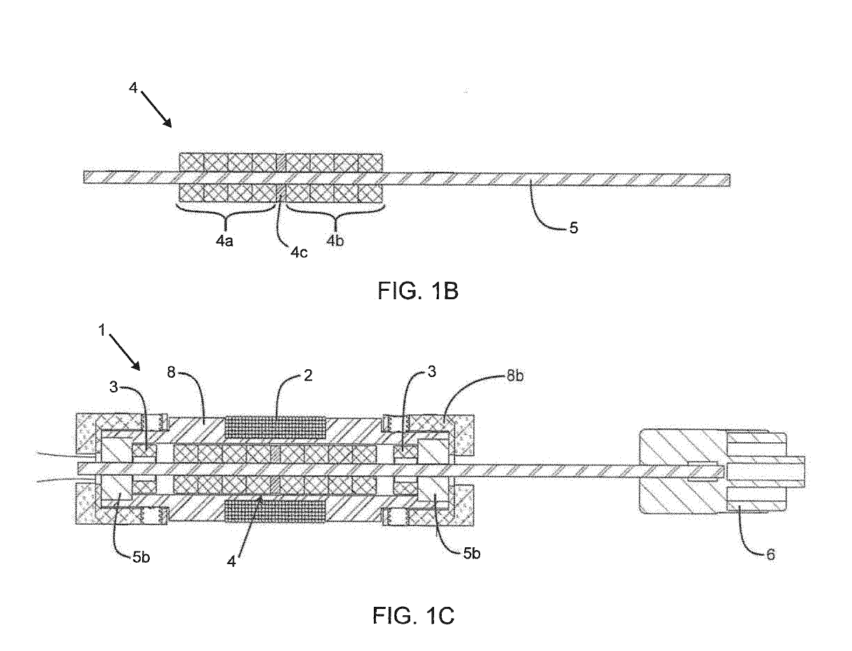

The preferred embodiments of the present invention are illustrated in FIGS. 1A-13 with the numerals referring to like and corresponding parts. For purposes of describing relative configuration of various elements of the Invention, the terms “distal”, “distally”, “proximal” or “proximally” are not defined so narrowly as to mean a particular rigid direction, but, rather, are used as placeholders to define relative locations which shall be defined in context with the attached drawings and reference numerals. A listing of the various reference labels are provided at the end of this Specification. In addition, as previously stated, U.S. Pat. Nos. 8,043,229 and 8,328,738 were incorporated by reference into the present application and include various embodiments.

The effectiveness of the invention as described, utilizes high-speed oscillatory motion to reduce forces associated with inserting a penetrating member through tissue or materials found within the body. Essentially, when tissue is penetrated by a high speed operation of a penetrating member portion of the device, such as a needle, the force required for entry as well as the amount of tissue deformation is reduced. A reciprocating penetrating member takes advantage of properties of high speed needle insertion, but because the displacement during each oscillatory cycle is small (typically <1 mm) it still enables the ability to maneuver or control the needle, such as to follow a non-linear insertion path or to manual advance the needle to a precise target.

To exploit the reduction of force effect, the medical device of the present invention is designed such that the penetrating distal tip portion attains a short travel distance or displacement at high speed, axially reciprocating at a specified frequency. Utilizing the various device configurations as described in the aforementioned embodiments, it has been determined that the reciprocating motion of the penetrating member may include a displacement for the motor shaft of the driving actuator between 0.1-2 mm, more preferably between 0.5-1.5 mm, at a frequency of between 50-500 Hz, but most preferably at 75-200 Hz for insertion into soft tissues within the body. This motion is caused by the penetrating member 10 being attached to a voice coil motor operated with an AC power signal.

Generally, any type of motor comprising an actuator assembly, further comprising a voice coil motor (VCM), or solenoid, or any other translational motion device, including piezoelectric actuators, would serve as a driving actuator and also fall within the spirit and scope of the invention.

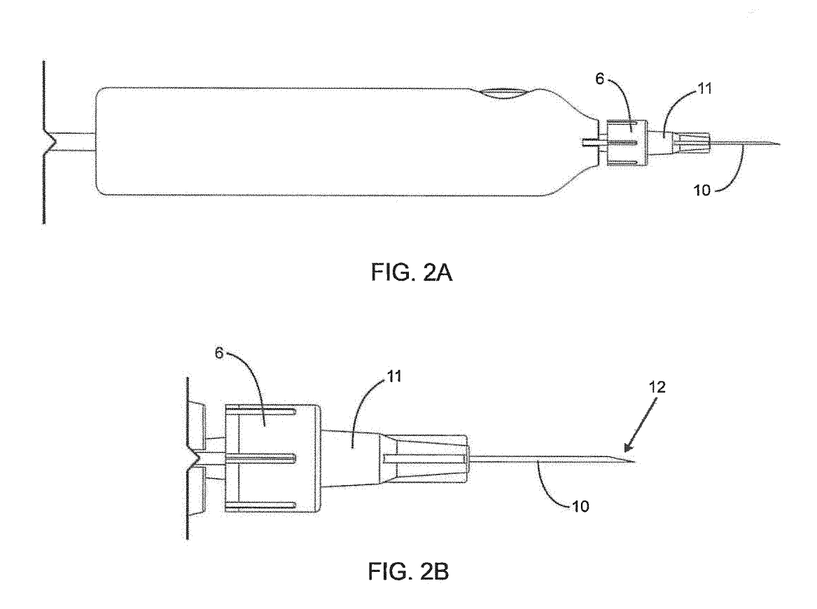

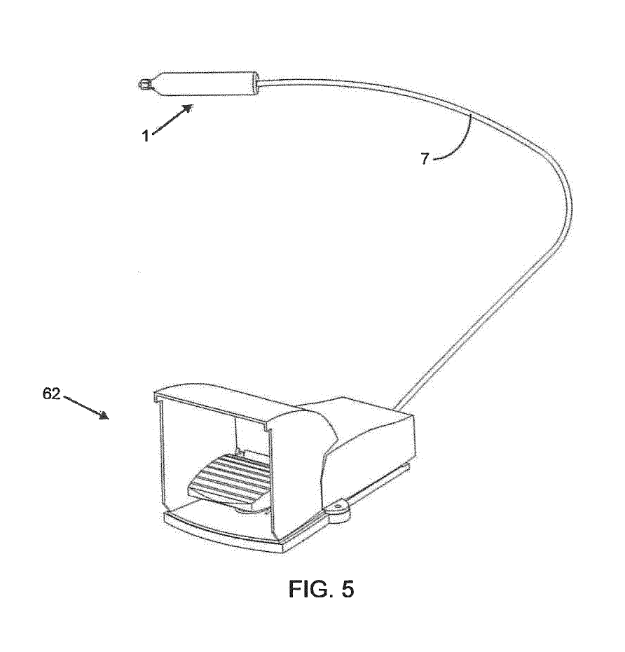

FIG. 1A depicts an embodiment of the present invention using a linear VCM as the mechanism for the driving actuator 1. FIG. 1A through 3C show cross-sectional view A-A 58, cross-sectional view of the magnet assembly 4, and a detail cross-sectional view of the VCM. A VCM creates low frequency reciprocating motion. In particular, when an alternating electric current is applied through the conducting voice coil 2, the result is a Lorentz Force in a direction defined by a function of the cross-product between the direction of current delivered by the power cable 7 (see FIG. 5) to the voice coil 2 and magnetic field vectors of the magnet arrays 4a and 4b. The two magnet arrays, 4a and 4b, have equal and opposing magnetic polarity vectors and are separated by a pole piece 4c. Together, the magnet arrays 4a, 4b, and pole piece 4c make up the magnet assembly 4. By alternating the direction of the current in the voice coil 2, a sinusoidal alternating force is applied to the magnet assembly 4 resulting in a reciprocating motion of the motor shaft 5 relative to the VCM body 8 which is seated inside the driving actuator handpiece body 1b. The VCM body 8 may be constructed of metal or of plastic with a low coefficient of friction. Delrin is a preferred material choice. The motor shaft bearings 5b provide supplemental friction reduction and help to ensure the motor shaft movement is directed solely in the axial direction (coaxial with the VCM body 8). The reciprocating motor shaft 5 communicates this motion to a keyed coupler 6 and attached penetrating member 10 (see FIG. 2A). The penetrating member 10 may be a hypodermic needle, a solid lancet, or other sharp and may be bonded to a hub 11 (see FIG. 2A) such as, but not limited to a LUER-slip or LUER-lok style. FIG. 2B depicts a close up view of the penetrating member 10 attached via a bonded hub 11 to the keyed coupler 6. The tip of the penetrating member 10 may have a bevel end 12 to increase sharpness.

Referring again to FIG. 1A, in all of the voice coil actuator configurations described, opposite polarity centering magnets 3 may be used to limit and control certain dynamic aspects of the driving actuator 1. At least one centering magnet 3 is located inside the VCM body 8 at each end. The centering magnets 3 have a same inward facing magnetic polarity as the outward facing polarity of the magnet assemblies 4a and 4b; the VCM end caps 8b keep the centering magnets 3 held in place against the repelling force. The opposition of magnetic forces (between centering magnets 3 and magnet assembly 4) acts to keep the magnet assembly centered at the midpoint of the VCM body 8. The magnets are placed at a certain distance from the ends of the magnet arrays 4a and 4b so that they are forced back toward center following peak displacement, but far enough away that no physical contact is made during oscillations. As with other voice coil embodiments using coils, the basic principle of actuation is caused by a time varying magnetic field created inside a solenoidal voice coil 2 when AC current flows in the coil wire, delivered via the power cable 7. The time varying magnetic field acts on the magnet arrays 4a and 4b, each a set of very strong permanent magnets. The entire magnet assembly 4, which is rigidly attached to the motor shaft 5, oscillates back and forth through the voice coil 2. The centering magnets 3 absorb and release energy at each cycle, helping to amplify the oscillating motion experienced by the penetrating member 10 (shown in FIGS. 2A and 2B). The resonant properties of the device can be optimized by magnet selection, number of coil turns in the voice coil 2, mass of the motor shaft 5, and by the elimination of frictional losses as much as possible (e.g. between the magnet assembly 4 and VCM body 8, or between the motor shaft 5 and motor shaft bearings 5b). Furthermore, performance can be optimized by adjusting the strength of the repelling force between the ends of the magnet arrays 4a and 4b and the opposing polarity centering magnets 3, thus modulating the stiffness and overall frequency response of the system. Friction is further eliminated by utilizing a ring style magnet for the centering magnets 3 whose inner diameter is sufficiently larger than the outer diameter of the drive shaft 5. Most application embodiments will require the magnets 3, 4a, and 4c to be made of a Neodymium-Iron-Boron (NdFeB) composition. However other compositions such as, but not limited to Samarium-Cobalt (SmCo), Alnico (AlNiCoCuFe), Strontium Ferrite (SrFeO), or Barium Ferrite (BaFeO) could be used. Slightly weaker magnets could be more optimal in some embodiments, such as a case where the physical size of the system is relatively small and strong magnets would be too powerful.

Feedback means via LVDT 69 and LVDT core 70 can be implemented to monitor oscillatory displacement magnitude, oscillatory frequency, and displacement magnitude from center position. Oscillatory displacement magnitude can be utilized as electromechanical feedback for ensuring the motor shaft 5 is displacing optimally and also potentially can provide a signal that triggers an auto-shut of mechanism. Additionally the LVDT 69 and LVDT core 70 can be used as a force sensor by monitoring the oscillatory center position and comparing it to the unloaded center position. The displacement from center position can be calibrated to relate to a force, since the restoring force provided by the centering magnets 3 increases in proportion to the displacement. This information can be relayed to the operator and/or used as an operating state change trigger.

In some embodiments where larger displacements are desired or a lower resonant frequency is needed, the function of the centering magnets 3 may be replaced with springs, elastic material, and may include a means to dynamically modulate the stiffness of the restoring force or to implement non-symmetric centering forces so that when the penetrating member experiences force from the tissue, the magnet assembly 4 would be located more centrally within the VCM body 8.

One aspect of performing procedures correctly is a manner in which to hold the bevel end (12 in FIG. 2B) of the penetrating member (10 in FIGS. 2A and 2B) rotationally stable. For example, during venipunctures for medication delivery, blood sampling, or for catheterization, a clinician will attempt to locate the tip of a small needle into the center of the vessel. Whether using a lancet or hypodermic needle, the standard technique is to ensure the bevel end (12 in FIG. 2B) of the penetrating member (10 in 2A and FIG. 2B) is “facing up” throughout the penetration event. This is generally not a problem while holding the needle directly in the fingers but needs to be taken into account when the needle is attached to the driving actuator (1 in FIG. 1A). Since the moving magnet assembly (4 in FIG. 1A) does not require leads to be run to the moving part of the motor, as is the case for moving coil actuators, the motor shaft (5 in FIG. 1A) is generally free to rotate within the VCM body (8 in FIG. 1A) meaning that the attached keyed coupler 6 that receives the hub 11 rotates freely. This minimizes frictional losses, but poses a problem for connecting a beveled penetrating member (10 in 2A and FIG. 2B) to the end of the motor shaft (5 in FIG. 1A) because the bevel is not rotationally stable throughout the penetration process. Using springs as the restoring force for centering the magnet assembly (4 in FIG. 1A), supplies some rotationally resistive forces.

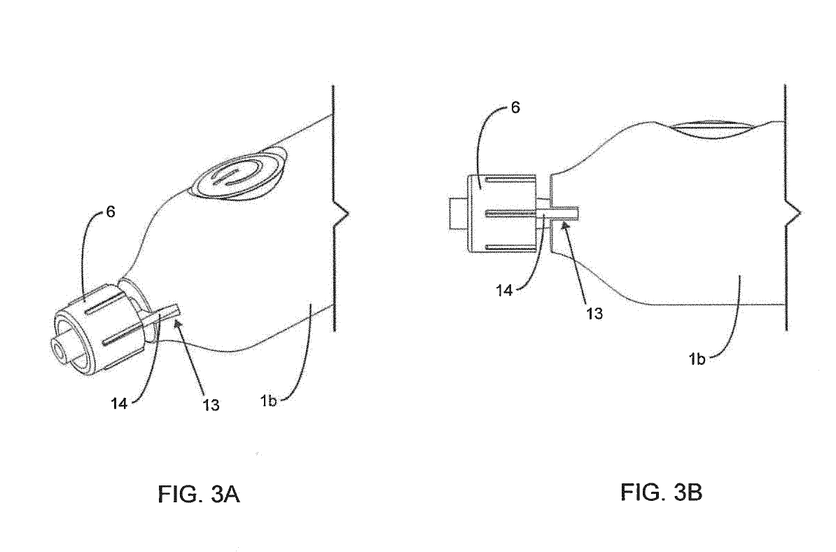

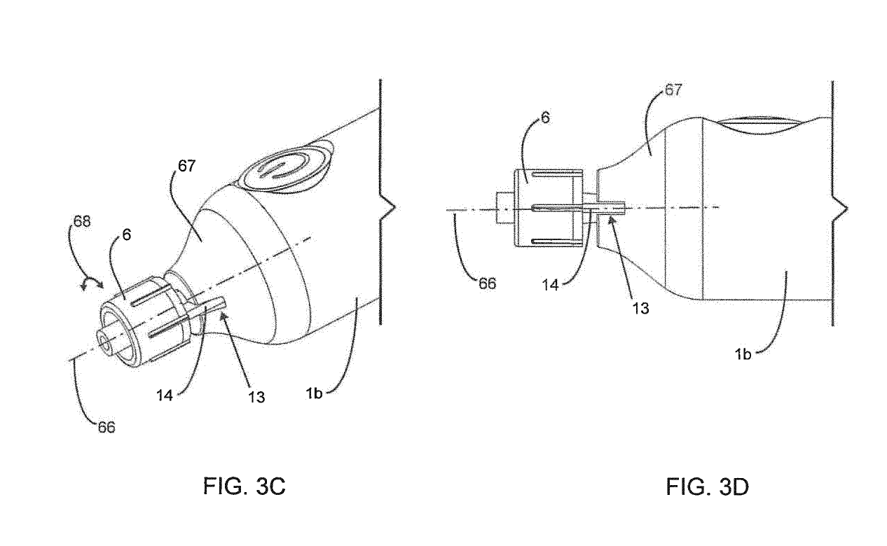

FIG. 3A presents one approach to restrict axial rotation of penetrating member (10 in FIG. 1C) when attached to the shaft (5 in FIG. 1A). A keyed coupler 6 with side tabs to serve as keys 14 is implemented in conjunction with keyway 13 formed by slots in the distal end of the driving actuator handpiece body 1b. The keyed coupler 6 is permanently fixed to the shaft 5 to allow reversible connection, for instance, to LUER-Lok needle hubs, but could be adapted for a range of other attachment schemes. FIG. 3B provides a lateral view of the coupling end of the driving actuator highlighting the keyed coupler 6 and surrounding keyway 13. Sufficient clearance between the keyway 13 slots on either side of the handpiece body 1b and the keys 14 is made to prevent frictional forces from damping out the oscillating motion. Friction can further be reduced between the keys 14 and keyway 13 by coatings and/or lining opposing surfaces with low friction materials. In an alternate embodiment depicted in FIGS. 3C and 3D, the front of the device incorporates a rotating keyway head 67 which can undergo controlled rotating motion 68 about a central axis of rotation 66. The motion may be produced by coupling the rotating keyway head 67, to rotational motor (not shown) such as a servomotor. This configuration would decouple the rotational and axial motions so that they can be controlled independently. The combined rotational and axial motions may further aid insertion especially into tougher tissues.

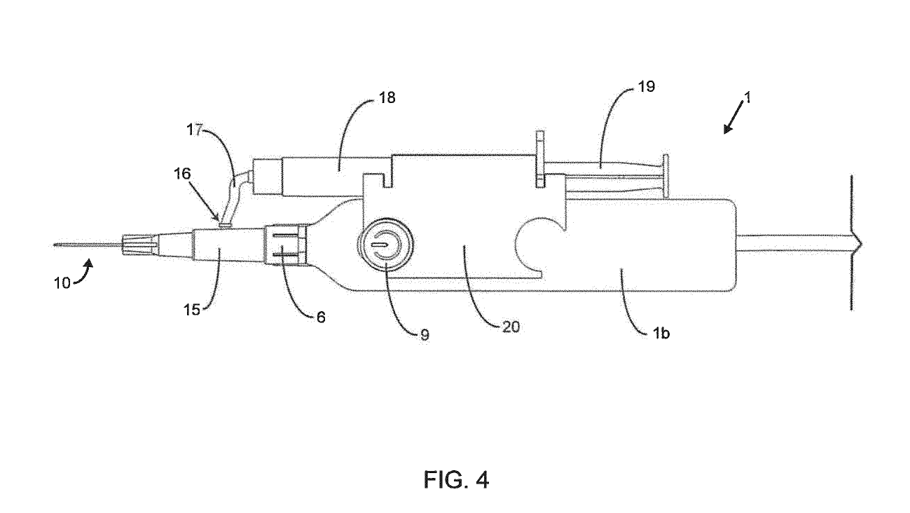

FIG. 4 shows an alternate embodiment of the device which incorporates a side port 16 which provides access to the inner lumen of the penetrating member 10. A segment of compliant tubing 17 may link the side port 16 to a fluid delivery source such as a syringe. The syringe body 18 can be reversibly attached to the driving actuator handpiece body 1b by a syringe coupling bracket 20. When the plunger 19 is pressed into the syringe body 18, fluid (such as medication, fluids, or vaccines) may be delivered into the body via an inner lumen of the penetrating member 10. In other applications, this or a similar embodiment would allow for extraction of fluids, tissue, or other materials (such as blood, fluid, or cells) into the syringe body 18 by pulling back on the syringe plunger handle 19 to create a negative pressure inside the compliant tubing 17 and inner lumen of the penetrating member 10. The compliant tubing 17 is sufficiently flexible so as not to impede the axially-directed oscillatory motion of the keyed coupler 6 or attached penetrating member 10. Obtaining inner lumen access may be implemented by attaching an intervening coupling piece with side port 15 between the fixed hub of the penetrating member 10 and the keyed coupler 6 as shown in FIG. 4, it could also be implemented by incorporating a side port directly into the fixed hub of the penetrating member 10. Further, the compliant tubing 17 could either be permanently integrated into the hub or coupling piece, or be an independent component with end fittings that reversibly mate with the side port 16 and syringe body 18. Other similar embodiments are envisioned that include a mounted syringe or other method of fluid injection into a side port 16, including gun-style injectors of vaccines and other medications for care and treatment of livestock in agricultural settings.

FIG. 5 presents another approach through use of a foot switch 62, to initialize and de-initialize power supplied to the driving actuator 1 via the power cable 7. This approach can also incorporate both the foot switch 62 and the power button 9 (not shown) for the option of initializing and de-initializing power to the driving actuator 1.

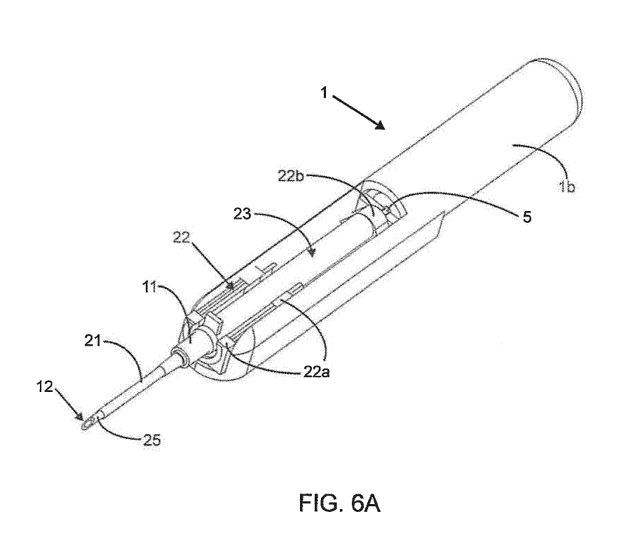

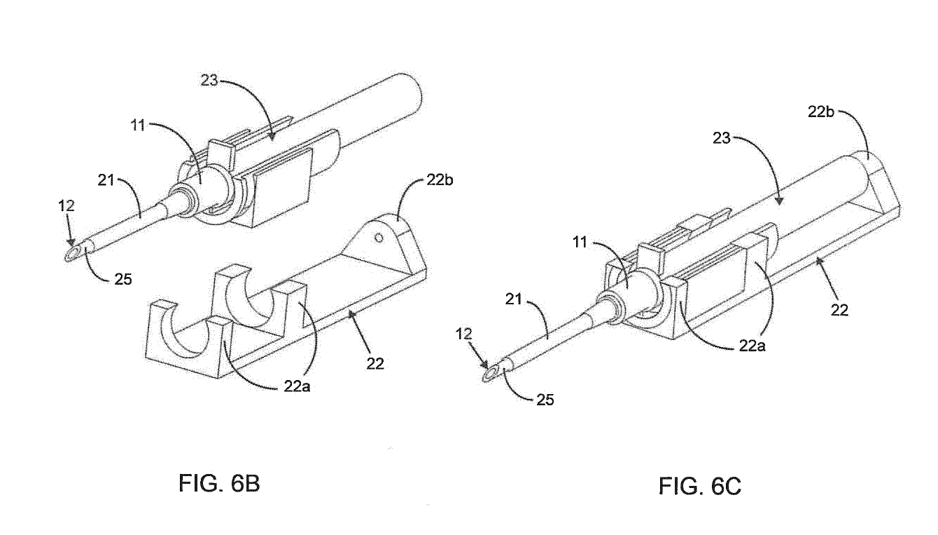

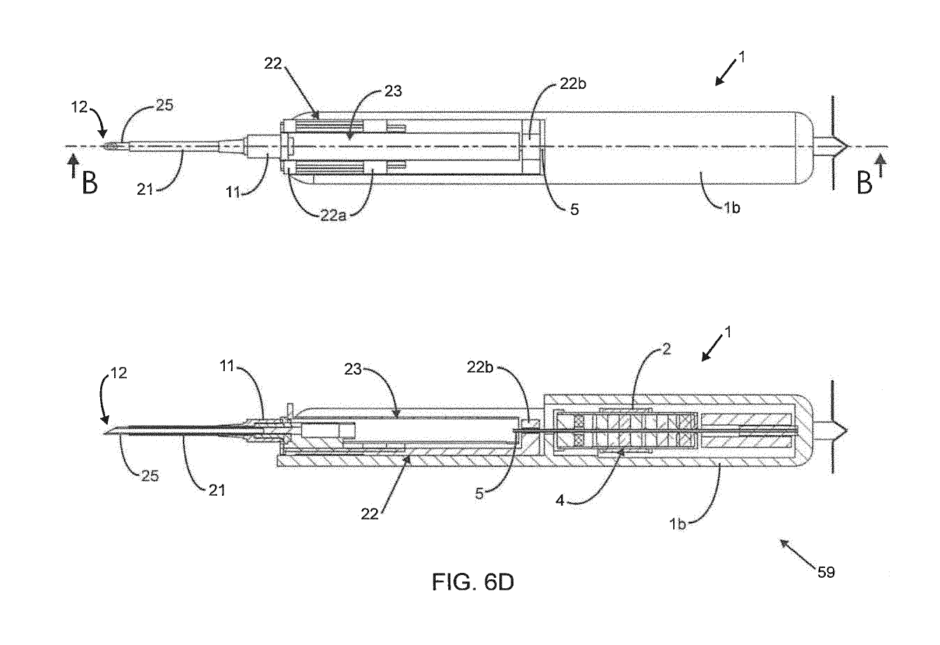

In another embodiment as shown in FIG. 6A-6D, the driving actuator 1 is used to aid the placement of an IV catheter into a vessel in order to have long-term access to the vascular system. This could be done by using a safety N device 23 or any other device with an attached penetrating member that does not have a hub that can be easily attached to the driving actuator 1. In this case the driving actuator 1 must be adapted to couple the motor shaft 5 to the body of the penetrating device. This requires the coupling to occur more from the lateral aspect of the device to be oscillated, rather than at the proximal end because a hub is not present or is inaccessible. To accomplish this, a coupling sled 22 (shown in more detail in FIGS. 6B and 6C) that has dips 22a that are geometrically compatible with specific penetrating devices is used to attach the penetrating device to the reciprocating motor shaft 5. The proximal end of coupling sled 22b connects to the motor shaft 5 which is forced back and forth by the interaction of the magnet assembly 4 and the magnetic field generated by electric current flowing through the voice coil 2. The coupling sled 22 is supported and guided by the structure of the handpiece body 1b. During a vascular access procedure, for instance, the driving actuator 1 delivers oscillatory motion to the IV penetrating member 25 to aid tissue penetration. When the bevel end 12 is inside the vessel to be catheterized, the IV catheter 21 is slid off the penetrating member 25 and into the vessel. The penetrating member 25 is then retracted into the body of the safety IV device 23, which can be removed from the clips 22a of the coupling sled and discarded. In FIG. 6C, the attachment of a safety IV device 23 to the coupling sled 22 is shown in isolation.

To ensure that the oscillatory motion is not over damped by the coupling sled 22, the moving mechanism must have sufficiently small resistance coefficient. In one embodiment the coupling sled is guided solely by the shape of the handpiece body (1b in FIG. 6D, section B-B 59). Here the interfacing surfaces are comprised of two materials having a low coefficient of friction. In another embodiment the coupling sled may be guided by for instance a linear ball-bearing guide rail. In another embodiment the coupling sled is capable of attaching to one or more linear round shafts utilizing bearings or material surfaces with low coefficient of friction to minimize sliding resistance.

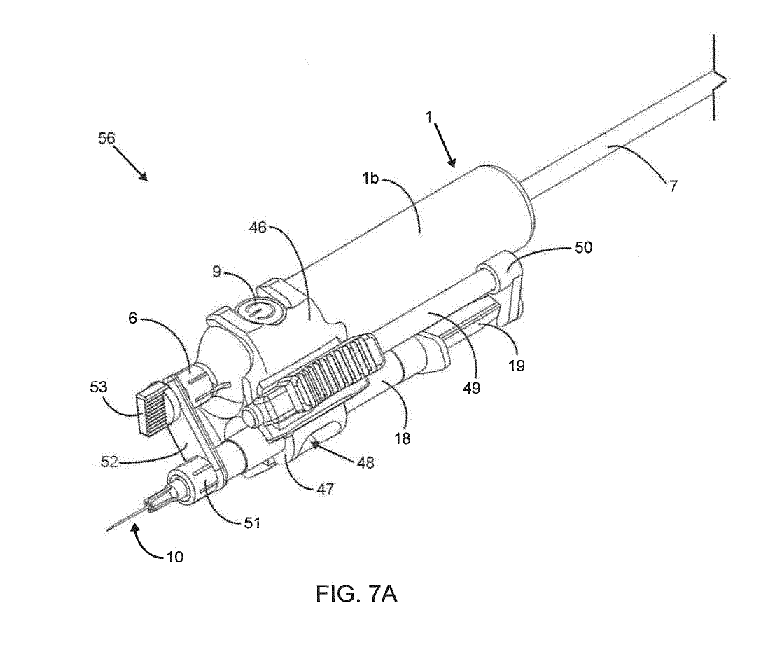

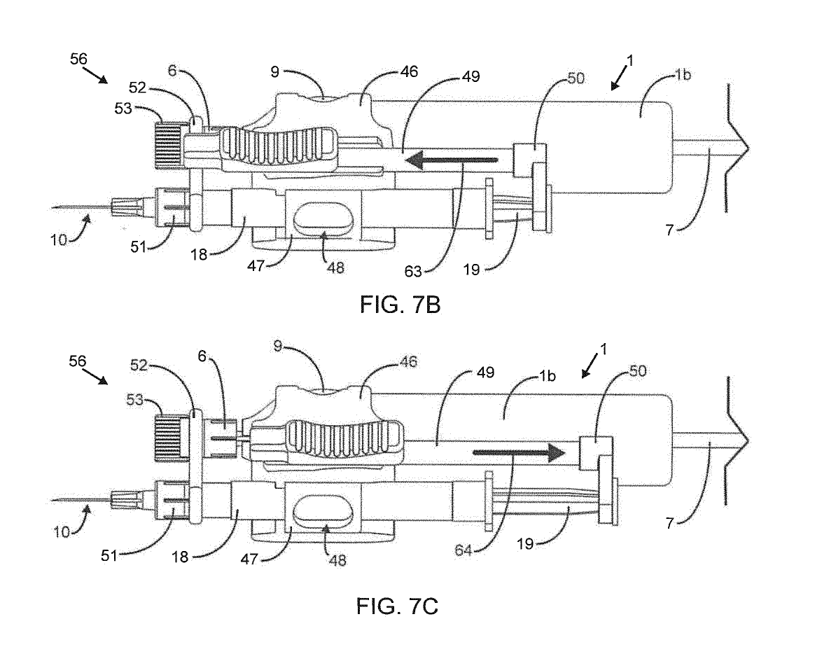

FIG. 7A-7C shows an alternate embodiment, slider device 56, which incorporates a fluid delivery source, such as a syringe, actuated by a driving actuator 1. Power is initialized and de-initialized by the power button 9 and supplied to the driving actuator 1 via the power cable 7. This could also be done with use of a foot switch 62 (as shown in FIG. 5). The actuation is transferred from the driving actuator 1 to the syringe via a keyed coupler 6 and a syringe clip 52. The syringe clip is mechanically attached to the keyed coupler 6 by use of a LUER-Lok coupling member (such as a thumb coupler 53). The syringe dip 52 pivots around the thumb coupler 53 360° to allow for quick attachment and detachment to the syringe coupler 51 which provides a mechanical attachment to both the syringe body 18 and the penetrating member 10. The syringe body 18 can be reversibly attached to the driving actuator handpiece body 1b by a handpiece clip 46. The syringe body 18 could be held in place using an interchangeable syringe adapter 47 that is inserted into a cavity of the handpiece dip 46, allowing for different sizes of the syringe body 18 and allowing for precise linear movement of the syringe body 18 within the syringe adapter 47. A means of visibility such as the syringe adapter window 48 is used to allow for clear visibility of the level of fluid (such as medication, fluids, or vaccines) within the syringe. When the plunger 19 is pressed into the syringe body 18, fluid may be delivered into the body via an inner lumen of the penetrating member 10 that is attached to the syringe body 18 through a syringe coupler 51. One-handed operation of the device can be achieved by allowing movement of the plunger 19 to be initiated through movement of the guide shaft 49 coupled to the plunger 19 through the guide shaft coupling 50. In other applications, this or a similar embodiment would allow for extraction of fluids, tissue, or other materials (such as blood, fluid, or cells) into the syringe body 18 by pulling back on the syringe plunger 19. A switch of the handpiece dip 46 may be located distal to the guide shaft coupling 50 and distal to some or all of the plunger 19. The switch of the handpiece clip 46 may be located adjacent the exterior handpiece body 1b and may allow for easier and more convenient actuation of the plunger 19 during use of the device.

FIG. 7B shows this embodiment with the guide shaft 49 pressing the plunger 19 to a forward position 63 following delivery of fluid contents (or the starting condition for fluid removal procedure). FIG. 7C shows this embodiment with the guide shaft 49 pulling the plunger 19 to a backward position 64 for the purpose of removing fluids (or the starting condition for fluid delivery procedure).

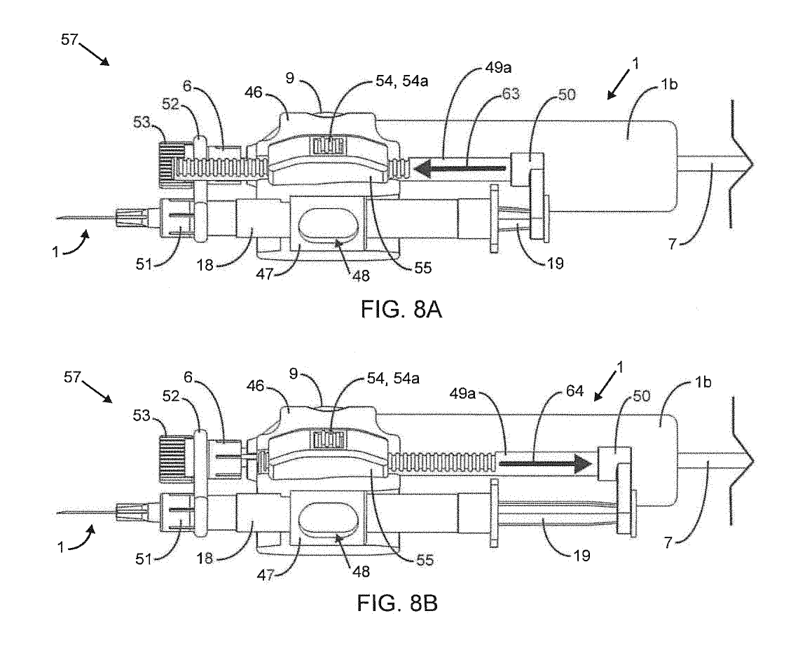

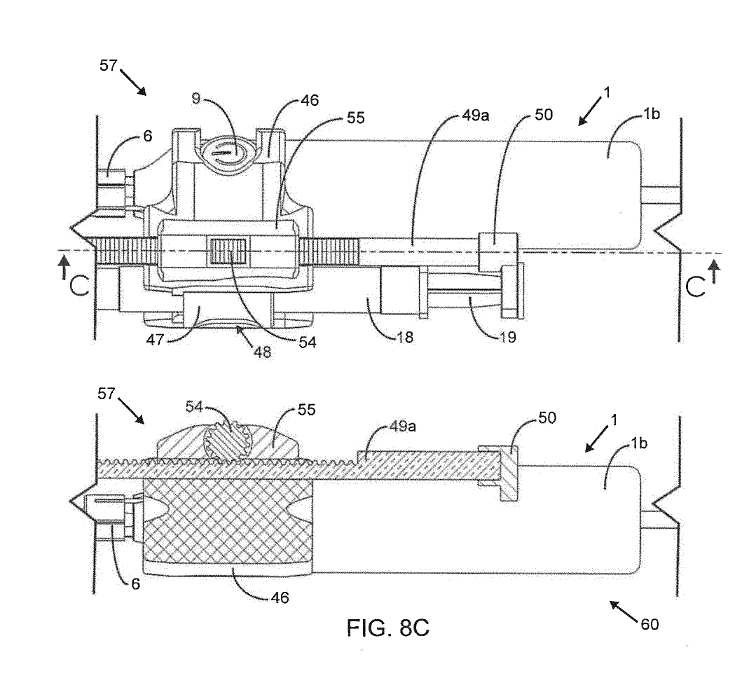

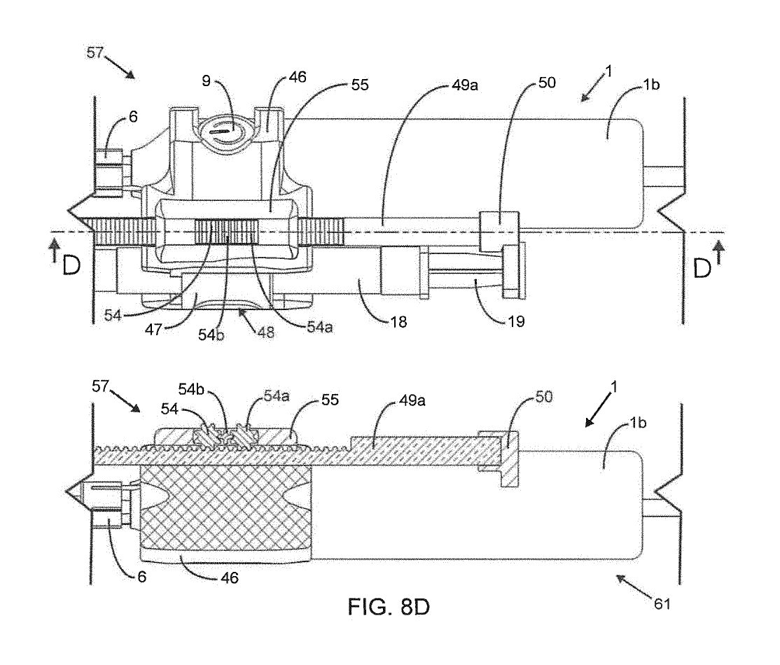

FIG. 8A-8C shows an alternate embodiment of FIG. 7A, geared slider device 57, which incorporates a fluid delivery source, such as a syringe, actuated by a driving actuator 1. Power is initialized and de-initialized by the power button 9 and supplied to the driving actuator 1 via the power cable 7. This could also be done with use of a foot switch 62 (as shown in FIG. 5). The actuation is transferred from the driving actuator 1 to the syringe via a keyed coupler 6 and a syringe clip 52. The syringe dip is mechanically attached to the keyed coupler 6 by use of a LUER-Lok coupling member (such as a thumb coupler 53). The syringe clip 52 pivots around the thumb coupler 53 360° to allow for quick attachment and detachment to the syringe coupler 51 which provides the mechanical attachment to both the syringe body 18 and the penetrating member 10. The syringe body 18 can be reversibly attached to the driving actuator handpiece body 1b by a handpiece clip 46. The syringe body 18 could be held in place using an interchangeable syringe adapter 47 that is inserted into a cavity of the handpiece clip 46, allowing for different sizes of the syringe body 18 and allowing for controlled linear movement of the syringe body 18 within the syringe adapter 47. The plunger 19 may move in relation to the handpiece body 1b. A means of visibility such as the syringe adapter window 48 is used to allow for clear visibility of the level of fluid (such as medication, fluids, or vaccines) within the syringe. When the plunger 19 is pressed into the syringe body 18, fluid may be delivered into the body via an inner lumen of the penetrating member 10 that is attached to the syringe body 18 through a syringe coupler 51. Movement of the plunger 19 is initiated through movement of the geared guide shaft 49a and is coupled to the geared guide shaft 49a through the guide shaft coupling 50. A mechanical mechanism including but not limited to a drive gear 54 or a drive gear accompanied by another gear, drive gear two 54a, housed within the drive gear housing 55 can be used to drive the geared guide shaft 49a. The means of providing forward or backward motion to the drive gear 54 or drive gear two 54a is through human kinetic energy or electric energy converted to mechanical energy such as but not limited to a DC motor (not shown). In other applications, this or a similar embodiment would allow for extraction of fluids, tissue, or other materials (such as blood, fluid, or cells) into the syringe body 18 by pulling back on the syringe plunger 19. FIG. 8A shows this embodiment with the geared guide shaft 49a pressing the plunger 19 to a forward position 63 following delivery of fluid contents (or the starting condition for fluid removal procedure). FIG. 8B shows this embodiment with the geared guide shaft 49a pulling the plunger 19 to a backward position 64 for the purpose of removing fluids (or the starting condition for fluid delivery procedure). FIG. 8C shows the geared slider device 57 with the use of a drive gear 54 to move the plunger 19 to a forward position 63 and a back position 64 as shown in FIGS. 8A and 8B. FIG. 8D shows the geared slider device 57 with the use of a drive gear 54 and drive gear two 54a to move the plunger 19 to a forward position 63 and a back position 64 as shown in FIGS. 8A and 8B. If only one gear is turned, drive gear 54 or drive gear two 54a, the other will move simultaneously do to the idler gear 54b along with the interlocking teeth of the geared drive shaft 49a.

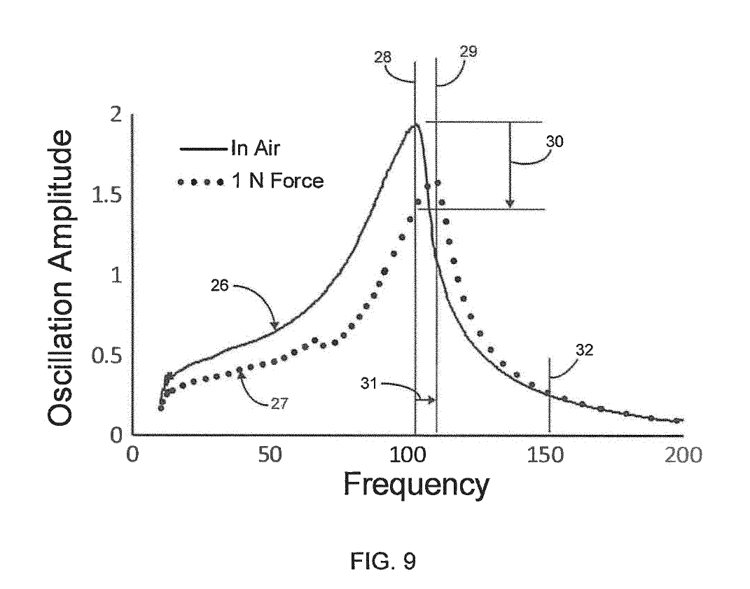

FIG. 9 displays experimental data obtained with a VCM embodiment of the driving actuator (1 in FIG. 1A) which demonstrates the frequency response behavior of the device as an elastic axial force is applied to keyed coupler 6 (not shown). The frequency response of the driving actuator in air (non-loaded) 26 exhibits resonant behavior with a peak displacement occurring at the resonant frequency in air 28. After the application of a moderate axial load of 1 N (simulating typical forces encountered during penetration of a 25 G hypodermic needle into rat tail skin), the device resonant frequency shifts 31 according to the new frequency response of driving actuator with axial force applied 27 (1 N elastic load force, applied axially). If the device were for instance operated at the original resonant frequency in air 28 when axial load force is applied during the course of tissue penetration, then it would cause an upward resonant frequency shift 31 with a resultant oscillatory displacement damping 30 at original resonant frequency 28. One method to overcome this shortcoming is to choose a damping resistant operating frequency 32 that is significantly higher than the original resonant frequency in air 28. As shown by the plots in FIG. 9, the damping effect of axial load on the oscillatory displacement amplitude is minimal at this damping resistant operating frequency 32, as shown by the overlap of the frequency response curves (i.e., frequency response on driving actuator in air (non-loaded) 26 and frequency response of driving actuator with axial force applied (loaded) 27) above this frequency.

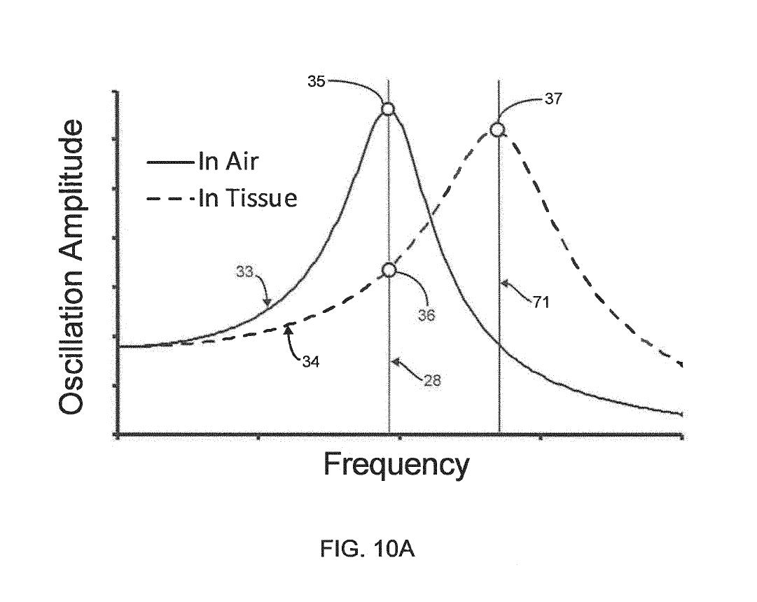

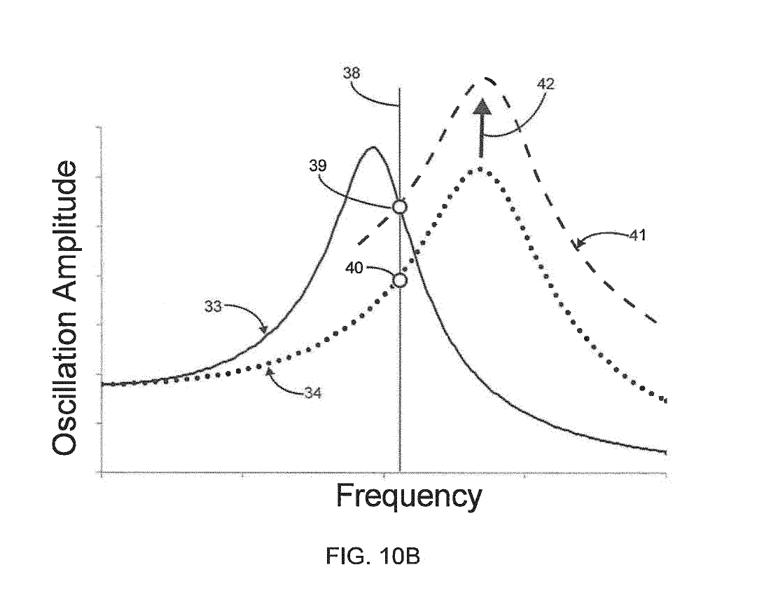

Another method of counteracting the oscillatory damping that is caused by the axial force applied to the penetrating member by the tissue is to employ feedback to adjust the operating frequency or current during the penetration. Two different approaches are now mentioned and illustrated with the aid of FIGS. 10A and 10B which show frequency response curves of a simulated 2nd order mass-spring-damper model with parameters chosen to match behavior comparable to driving actuator characterized in FIG. 9. The simulated frequency response in air 33 of a VCM-based driving actuator in air (non-loaded condition) has a resonant displacement peak in air 35 occurring at the resonant frequency in air 28. When the effect of elastic tissue interaction with the penetrating member is added to the model (as an increase in spring stiffness), the simulated frequency response in tissue 34 is shifted relative to the original simulated frequency response in air 33. The resonant displacement peak in tissue 37 occurs at a different, in this case higher, resonant frequency in tissue 71. The end result is a displacement in tissue at original resonant frequency 36 that is reduced because the resonant frequency in air 28 is different than the resonant frequency in tissue 71. In an embodiment employing a displacement sensor (e.g. LVDT) to monitor oscillatory displacement of the motor shaft 5 (not shown), the reduced displacement is sensed and the controller would adjust the operating frequency closer to the resonant frequency in tissue 71 so that the displacement would necessarily increase closer to the resonant displacement peak in tissue 37. By employing a feedback loop to continually adjust the operating frequency so that it is always near the current resonant frequency of the combined driving actuator-tissue system, power consumption of the device can be minimized.

In FIG. 10B, a second method of employing feedback to adjust driving parameters is depicted based on current amplitude control. In this method, current instead of frequency is adjusted during tissue penetration in an attempt to maintain oscillatory displacement levels. As an example, a driving actuator with simulated frequency response in air 33 is driven at the shown operating frequency 38 yielding the oscillatory displacement at operating frequency in air 39. When the penetrating member attached to the driving actuator contacts tissue, the simulated frequency response in tissue 34 may be shifted relative to the simulated frequency response in air 33 as the graph suggests. The shifted simulated frequency response in tissue 34 has reduced displacement at operating frequency after contacting tissue 40 at the operating frequency 38. To counteract the damping of displacement, current amplitude supplied to the driving actuator is increased, resulting in a modified frequency response following increase in current 41, shifted upward as indicated by the arrow 42. Current is increased until the oscillatory displacement reaches the displacement at operating frequency in air 39. At this point the modified frequency response 41 of the coupled system intersects the original simulated frequency response in air 33 at the operating frequency 38, albeit requiring a higher driving current amplitude.

Additional means for maintaining oscillatory displacement level could employ a combination of frequency and current control.

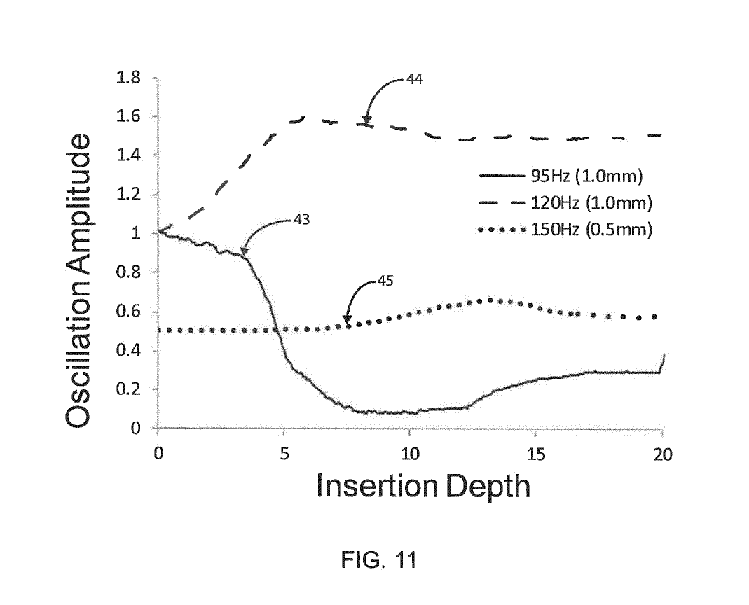

FIG. 11 shows the oscillatory displacement amplitude that was measured during insertions into skin tissue at different operating frequency. The resonant frequency of the driving actuator which was used to obtain these curves was near 95 Hz. When the operating frequency was chosen to coincide with the resonant frequency, the oscillatory displacement is damped considerably as shown in the displacement versus insertion depth plot with operating frequency at 95 Hz 43. Choosing an operating frequency of 120 Hz (25 Hz above resonant frequency), the displacement actually increases as the penetrating member contacted and inserted through tissue as shown in displacement versus insertion depth plot with operating frequency at 120 Hz 44. Choosing an even higher operating frequency, the displacement versus insertion depth plot with operating frequency at 150 Hz 45 remained relatively flat. Note: a smaller starting displacement was chosen for plot 45 as compared to plots 43 and 44. Another notable feature with operating at a frequency above the resonance of non-loaded system is that the displacement tends to increase during penetration as the tissue adds axial force to the tip of the penetrating member as seen in plots 44 and 45. When this axial force is removed or reduced, such as when a vessel wall or tissue plane is penetrated, the displacement may decrease, reducing the risk of over penetration. When a feedback loop is employed to control the displacement (see descriptions of FIGS. 6A and 6B), abrupt changes in the axial force (e.g. penetration through a vessel wall) could be sensed by a change in driving characteristics (e.g. power, phase, resonant frequency, oscillation amplitude) to indicate needle tip location (e.g. entry into vessel lumen).

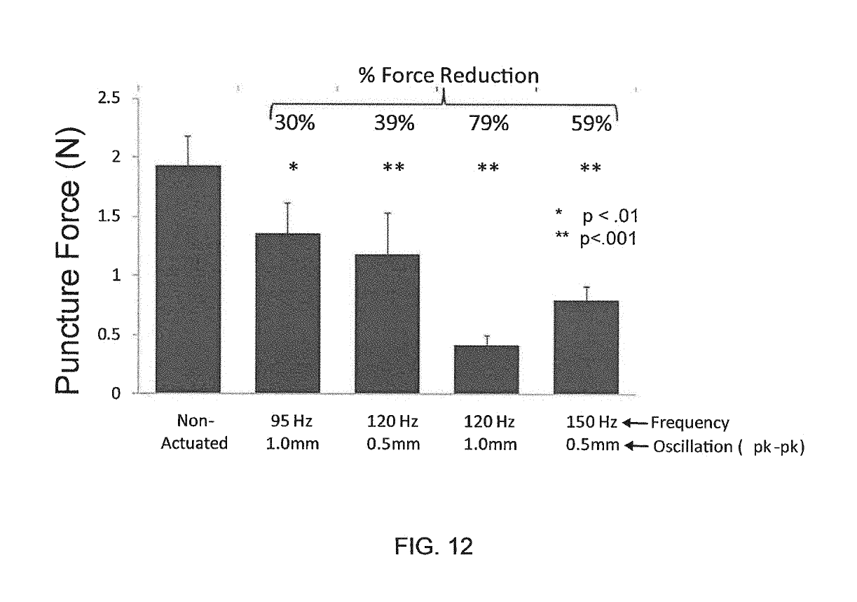

FIG. 12 presents data obtained from insertions into porcine skin with an 18 gauge hypodermic needle serving as the penetrating member. Performance for different operating frequency and starting (in air) oscillatory displacement settings are shown. Depending on the choice of operating parameters, significant force reductions are seen in comparison to insertions of a non-actuated (non-oscillated) needle.

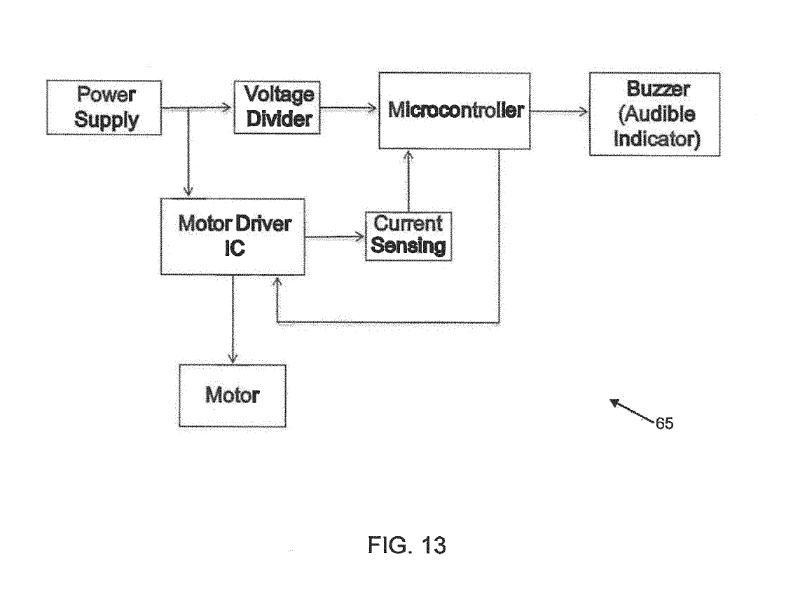

FIG. 13 is a control electronics diagram 65 that presents one method of utilizing voltage and current sensing for various control actions. The control electronics employ two sensing methods to ensure that the motor function is operating correctly and to signal the operator if any faults occur. The voltage from the power supply is applied directly to the Motor Driver IC. This voltage is also sensed by the Microcontroller through a Voltage Divider circuit. The Microcontroller monitors this voltage signal and will disable the Motor Driver IC and initiate the Buzzer if the voltage level is outside of a predetermined window. Likewise the Microcontroller also senses and monitors the current through the motor via a current sense pin on the Motor Driver IC. If this current level exceeds a predetermined limit the Microcontroller will disable the Motor Driver IC and initiate the Buzzer. In alternate designs the microcontroller could also be monitoring voltage and current frequency and their relative phase angles.

In the preferred embodiment of the VCM-based driving actuator 1, the VCM coil 2 may be driven by control circuitry such that a constant supply voltage can be applied to the VCM coil 2 at both positive and negative potential or can be turned off to apply zero volts. This supply voltage is switched on and off at a frequency between 10 kHz and 40 kHz where the time that the supply voltage is either ‘on’ or ‘off’ can be adjusted. The average voltage seen by the VCM coil 2 over a given switching cycle is proportional to the time the supply voltage is applied. For example, if the supply voltage is applied for 50% of the switching cycle the average voltage seen by the VCM coil 2 will be 50% of the supply voltage. When the VCM coil 2 is supplied with a positive potential voltage a force proportional to the applied voltage will be applied to the magnet assembly 4 of the VCM in one direction while a negative potential voltage will apply a force to the magnet assembly 4 in the opposite direction. By periodically reversing the polarity of the applied potential of the switching signal at 50-500 Hz, an oscillating force can be applied to the motor shaft 5 by way of the attached magnet assembly 4 with an average magnitude proportional to the average voltage magnitude of the generated signal. The energy of this signal will be located at the frequency at which the potential is reversed and every odd multiple of this frequency, the magnitude of which will decrease with each Increasing multiple. Likewise, additional energy will also be located at the switching frequency and every odd multiple of this frequency, the magnitude of which will decrease with each increasing multiple.

The frequency response seen in FIGS. 9, 10A and 10B is highly resonant with a weaker response far from the resonant frequency. When the actuator is driven with the described signal where the potential reversal frequency is near resonance, the effects of the energy at higher frequencies is greatly attenuated to the point that they are almost non-existent. This results in a very sinusoidal response without the need for additional filtering or smoothing circuitry. Driving the actuator using this method was chosen because the circuitry necessary to create the signal described is very simple, efficient and cost effective compared to sinusoidal signal generation and is able to take advantage of the physics of the actuator. The ability to use this method is one of the benefits of the VCM design because this method would not be practical to drive an actuator with a wide frequency response when only one frequency of actuation is desired.

Now that exemplary embodiments of the present invention have been shown and described in detail, various modifications and improvements thereon will become apparent. While the foregoing embodiments may have dealt with the penetration through skin, bone, veins and ligaments as exemplary biological tissues, the present invention can undoubtedly ensure similar effects with other tissues which are commonly penetrated within the body. For example there are multiplicities of other tools like central venous catheter introducers, laparoscopic Instruments with associated sharps, cavity drainage catheter kits, and neonatal lancets, as well as procedures like insulin administration and percutaneous glucose testing, to name a few, where embodiments disclosed herein comprising sonically or ultrasonically driven sharps members may be used to precisely pierce or puncture tissues with minimal tinting.

### Additional Embodiments

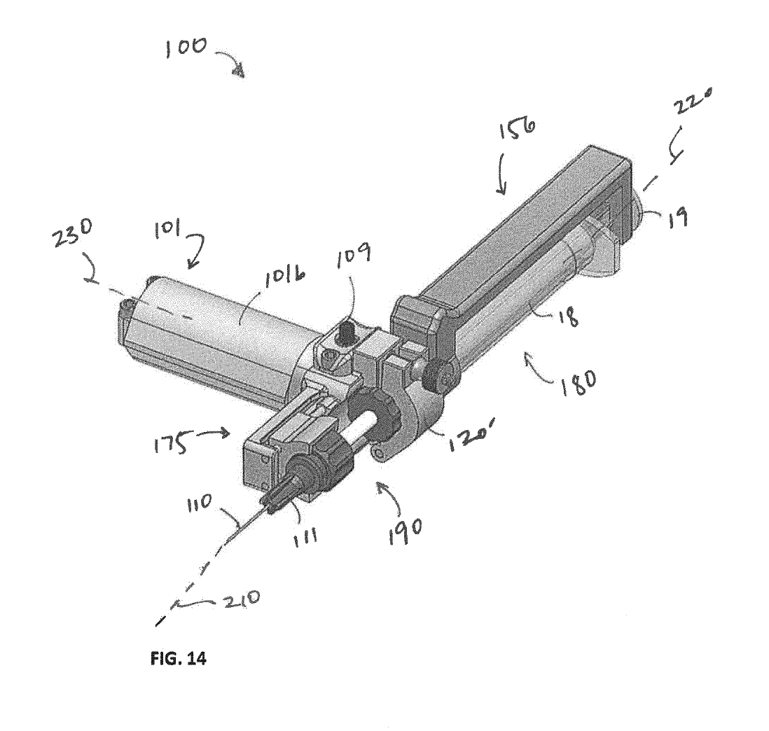

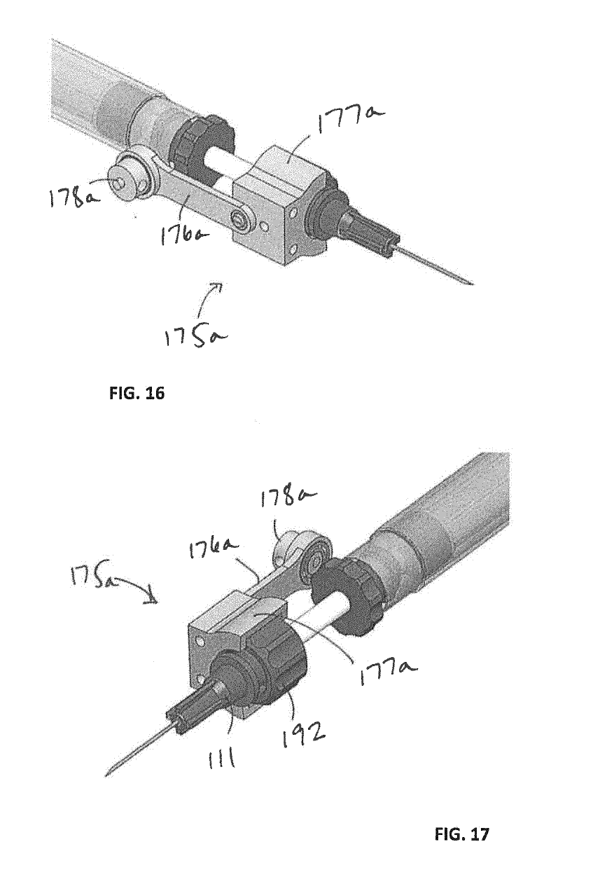

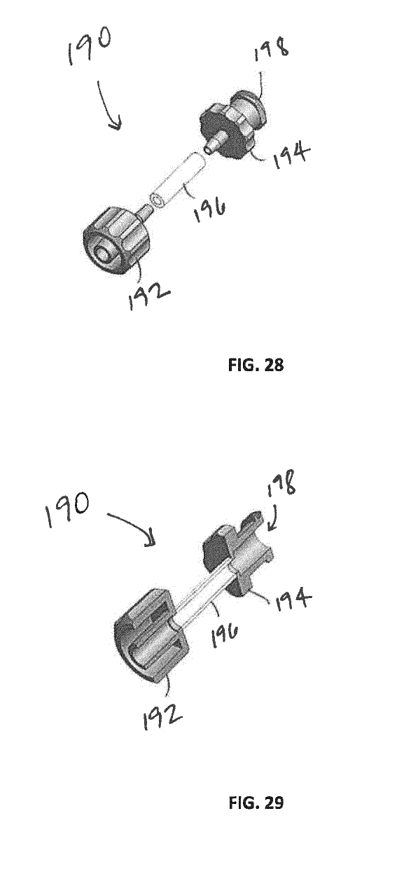

Further embodiments of the invention are shown in FIGS. 14-32. Such embodiments of the device may be referred to herein as an oscillating needle insertion device 100. As shown throughout FIGS. 14-32, these embodiments Include a penetrating member 110 as described above. For instance, and as shown in FIG. 14, the penetrating member 110 has a distal end that is sharp for insertion into tissue of a patient, and an opposite proximal end for connection to the remainder of the device 100, such as to a hub 111 as previously described. As before, the proximal end of the penetrating member 110 and the hub 111 may be selectively attachable to one another to enable removal when desired, or may be integrally attached for permanent connection.

The penetrating member 110 also includes a lumen extending therethrough between the distal and proximal ends. This lumen is dimensioned to receive and transmit fluid through the penetrating member 110, such as but not limited to blood in the case of blood draws or medications and/or saline in the administration of the same. Accordingly, the penetrating member 110 is configured to interconnect in fluid communication with a reservoir 180. The reservoir 180 may be any source, repository or space for receiving and/or holding fluids. For instance, in some embodiments the reservoir 180 may be a syringe having a syringe body 18 and plunger 19, as shown in FIG. 14. Such a reservoir 180 may be used both in collecting fluids such as blood from a patient or animal and in delivering fluids such as medication to a patient or animal.

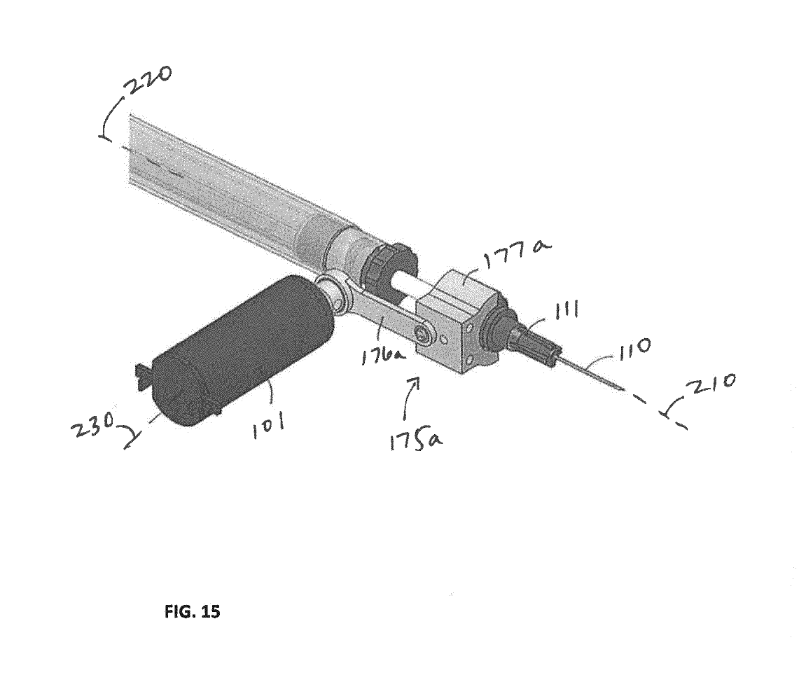

As can be appreciated from FIGS. 14 and 15, the penetrating member 110 defines a penetrating axis 210 along its length from the distal to proximal ends, and along which the lumen extends. The reservoir 180 may also define a reservoir axis 220 along its length, such as along the length of a syringe body 18 in the case of a syringe. In at least one embodiment of the present device 100, as in FIGS. 7A-8D, 14 and 15, the penetrating axis 210 and reservoir axis 220 may be coaxial with one another. In other embodiments, such as shown in FIG. 4, the penetrating axis 210 and reservoir axis 220 may be parallel to one another. In still other embodiments, the penetrating axis 210 and reservoir axis 220 may be at an oblique angle relative to one another. As used herein, “oblique” refers to any angle other than a perpendicular or parallel angle.

The device 100 also includes a driving actuator 101 which provides oscillating or reciprocating motion to the penetrating member 110. As used herein, “oscillating” and “reciprocating” may be used interchangeably to mean movement back and forth in a repetitive fashion. The device 100 may also include a power button 109 configured to activate and deactivate the driving actuator 101. It should be appreciated that the power button 109 may be a button, lever, pedal, keypad, or any other interface for turning the driving actuator 101 on and off. In some embodiments, as in FIG. 14, the power button 109 may be adjacent to the driving actuator 101 to facilitate one-handed operation of the device 100. Indeed, in some embodiments the driving actuator 101 is housed within a handpiece 101b that may be grasped by a user of the device 100 for use. The power button 109 may be located on the exterior surface of the handpiece 101b, or may be adjacent to the handpiece 101b.

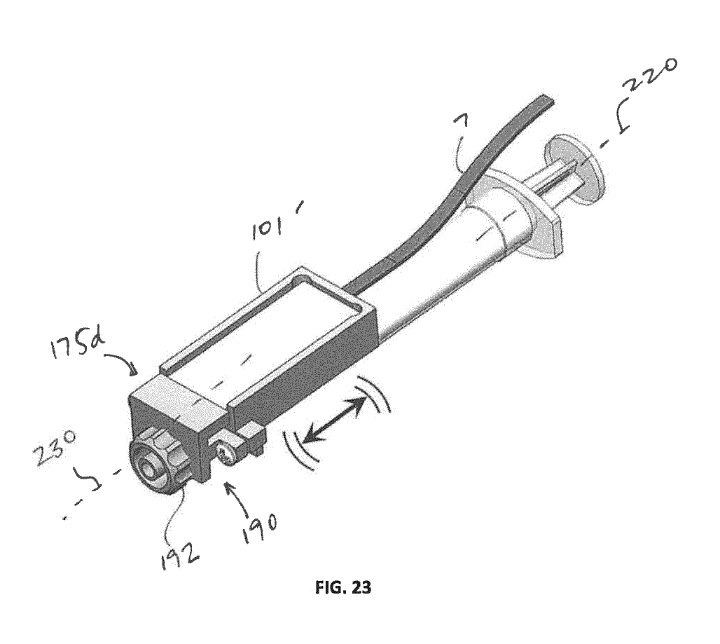

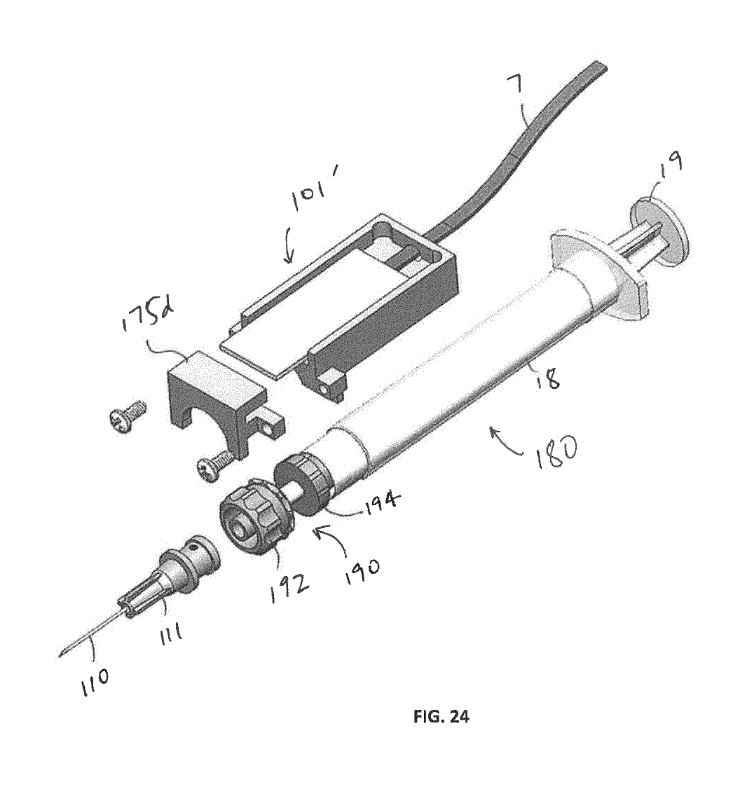

The driving actuator 101 may be a DC motor, piezoelectric element, voice coil motor, flextensional transducer or other motor as described in detail above. For example, as shown in the embodiments of FIGS. 14, 15 and 18-20, the driving actuator 101 may be a DC motor configured to generate rotational motion about a driving axis 230 when electrically activated. In other embodiments, as in FIGS. 23-24, the driving actuator 101′ may be a piezoelectric element such as a piezoelectric transducer configured to generate linear reciprocating motion as described previously along the driving axis 230.

The device 100 may also include a controller in electrical communication with the driving actuator 101 that is configured to operate the driving actuator 101 as described above. For instance, the controller may operate the driving actuator at a preselected frequency which may be selected based on the particular tissue to be penetrated. The chosen preselected frequency may be at or near a resonant frequency of the penetrating member 110 in the desired target tissue, or may be chosen as sufficient to offset the damping of oscillations that occurs upon moving from one medium to another such as from air to tissue or from one tissue to another. In some embodiments, the preselected frequency is higher than a resonant frequency in tissue or air, and in some embodiments, may be in the range of ⅓ to ½ an octave higher than the resonant frequency in air. In other embodiments, the controller may variably adjust the operating frequency during use of the device 100 based on feedback in order to maintain the operating frequency at or near a resonant frequency of the penetrating member 110 in the target tissue. All of this is as described in greater detail above.

In still other embodiments, the controller is configured to operate the driving actuator 101 at optimal driving parameters for the particular type and/or model of driving actuator 101. For instance, DC motors may be controlled by varying the current and voltage which dictates frequency and torque. Changing one parameter affects the values of the other parameters. Each type of DC motor may have a set or range of operating parameters that may be known (such as from the manufacturer) to provide optimal performance. This set of parameters are referred to herein as the optimal driving parameters. For example, DC motors may be operated in the range of 3-24 volts for voltage, 50-1000 Hz for frequency, and at least 0.3 mNm for torque. In at least one embodiment, the DC motor may be operated at 12 volts voltage, 100-160 Hz frequency, and 0.45 mNm torque as the optimal driving parameters for a Faulhaber 1506N012SR DC motor (manufactured by DR. FRITZ FAULHABER GMBH & CO. KG, Schönaich, Germany), although other types of DC motors may also be used.

The motion from the driving actuator 101 is transferred to the penetrating member 110 through a motor linkage 175. As show in FIGS. 14-24, the motor linkage 175 interconnects the driving actuator 101 with the penetrating member 110. The components of the motor linkage 175 may be made of rigid construction but connected to permit movement, so that motion generated by the driving actuator 101 is conveyed to the penetrating member 110 and results in the penetrating member 110 linearly reciprocating along the penetrating axis 210. Accordingly, the driving actuator 101 is configured to linearly reciprocate the penetrating member 110.

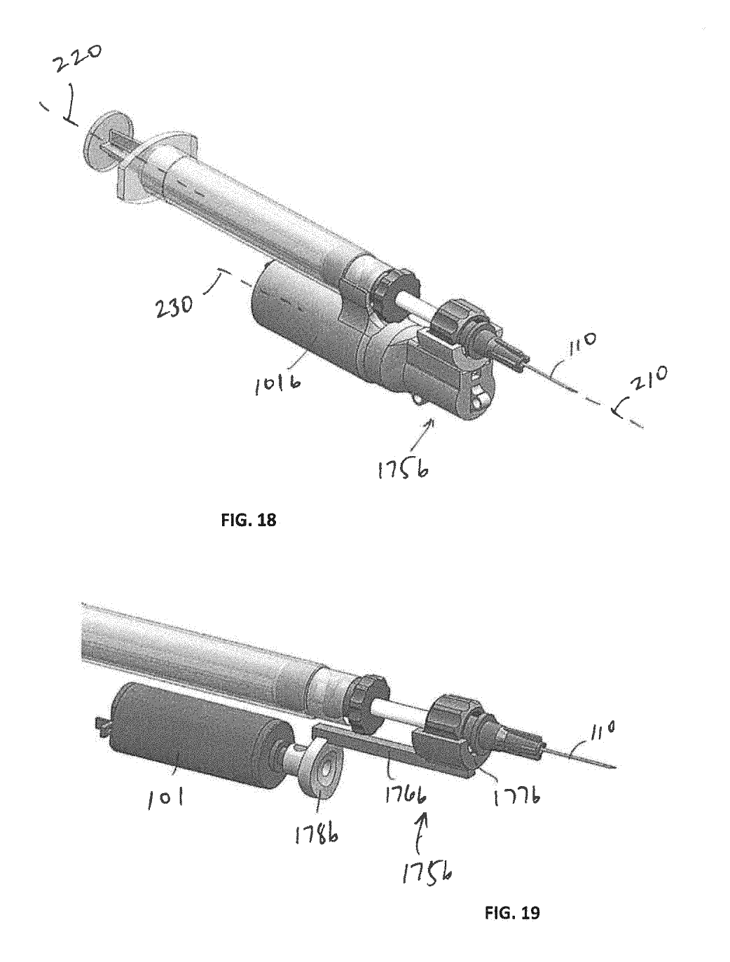

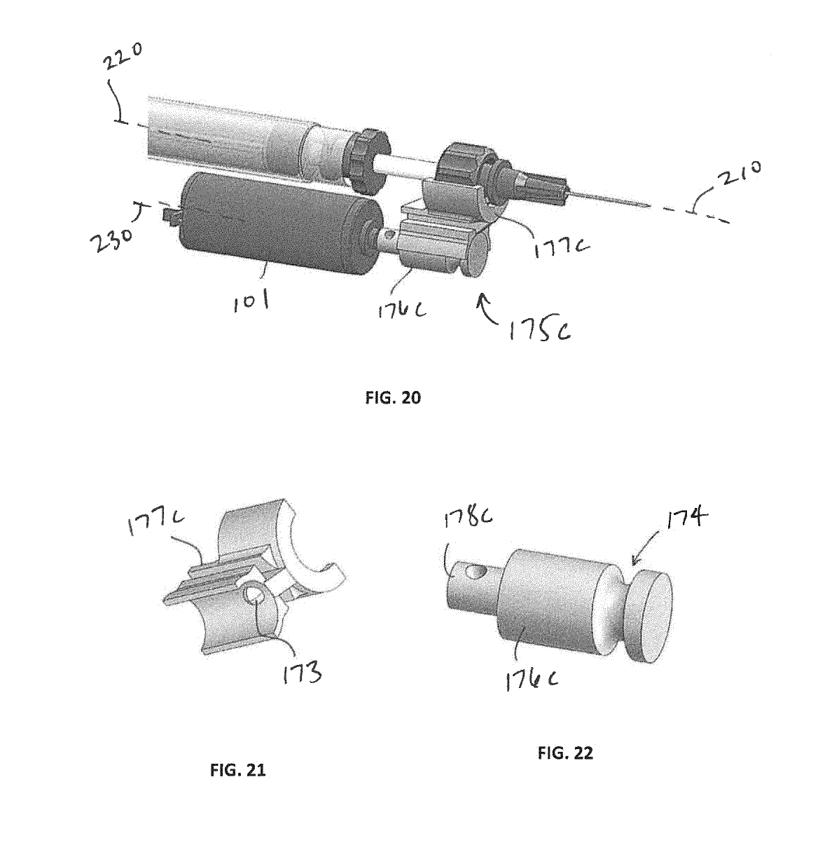

To facilitate this conveyance of motion, the motor linkage 175 may include a number of component parts. For instance, the motor linkage 175 may include a motor connection 178, which is dimensioned to connect directly with a portion of the driving actuator 101. The motor connection 178 may be a pin, socket, ball, or any suitable connection shaped or configured to engage the driving actuator 101. In some embodiments as in FIGS. 14-17, the motor connection 178a provides a mobile connection point, such as a pivot point or joint, that accepts and moves with the rotational motion of the motor 101. This may be a rotational pivot point in a slider crank mechanism or scotch yoke mechanism. In other embodiments, as in FIGS. 18-19, the motor connection 178b may be a swash plate or other similar structure that rotates with the rotational motion of the driving actuator 101. In still further embodiments, as in FIGS. 20-22, the motor connection 178c may be a terminal portion of a barrel cam or other similar structure that connects with the rotational motor.

The motor linkage 175 may also include an extension portion 176 that extends from the motor connection 178. The extension portion 176 is of rigid construction, is preferably linear, and has a length that substantially spans the distance between the driving actuator 101 and the penetrating member 110. The extension portion 176 may be fixed at one end and permit rotational motion at the other end to convert the rotational motion into linear motion. For instance, in the embodiment of FIGS. 14-17, the extension portion 176a is attached to and extends from the motor connection 178a opposite end from the motor 101, and translates the rotational motion of a DC motor into linear motion along the length of the extension portion 176a. In certain embodiments, the motor connection 178a and extension portion 175a together may form a slider crank mechanism. In other embodiments, the motor connection 178a and extension portion 175a may form a scotch yoke mechanism. These are a few non-limiting examples, and any mechanism of converting rotational motion to linear motion may be used for the motor linkage 175. In other embodiments, as in FIGS. 18-19, the extension portion 176b may be a rod fixedly connected to or integral with the motor connection 178b, which may be a swash plate. In still further embodiments, as in FIGS. 20 and 22, the extension portion 176c may constitute a barrel cam where one end is the motor connection 178c. These are a few non-limiting examples.

The motor linkage 175 may also include a coupler 177 that connects the extension portion 176 with the penetrating member 110 to convey the linear motion the penetrating member 110. The coupler 177 may attach to the extension portion 176 at the opposite end from the motor connection 178. For Instance, in the embodiment of FIGS. 14-17, the coupler 177a may be formed as a clip that connects to the penetrating member 110, hub 111, or other component of the device 100 that may be proximal to the penetrating member 110 by snap-fit or other selective attachment. In a preferred embodiment, the coupler 177a is rigidly affixed to the extension portion 176a of the motor linkage 175a such that the linear motion of the extension portion 176a is transferred to the penetrating member 110. One end of the extension portion 176a may be secured to the coupler 177a, such as with a screw, pin, or adhesive. In other embodiments, as in FIGS. 18-19, the coupler 177b may attach to the extension portion 176b by adhesive or may be bonded or integrally formed therewith. In still further embodiments, as in FIGS. 20-22, the coupler 177c may include a protrusion 173 that extends from the coupler 177c and is configured to be received and movably retained within a matching groove 174 of the extension portion 175c. This interaction between the groove 174 and protrusion 173 converts the rotational motion of the barrel cam extension portion 176c to linear/translational motion.

In still further embodiments, as in FIGS. 23 and 24, the motor linkage 175d may include the motor connection, extension portion and coupler all within a single piece. Such embodiments may be useful for more direct connection between the driving actuator 101′ and the penetrating member 110. For Instance, a piezoelectric motor as the driving actuator 101′ produces linear vibrations that may be transferred directly to the penetrating member 110 without the need to convert them. A simpler motor linkage 175d transmits this motion without conversion.

The motor linkage 175, and more specifically the extension portion 176, may extend in any direction relative to the driving axis 230 of the driving actuator 101. For example, as shown in the embodiments of FIGS. 14 and 15, the motor linkage 175a extends perpendicular to the driving axis 230 of the driving actuator 101. In such embodiments, the driving actuator 101 may be a rotational motor such as a DC motor which creates rotational motion about the driving axis 230. The motor linkage 175a converts this rotational motion to linear or translational motion in a direction perpendicular to the driving axis 230, such as with a slider crank or scotch yoke mechanism of motor linkage 175, so the penetrating member 110 reciprocates along a penetrating axis 210 that is perpendicular to the driving axis 230. In other embodiments, as in FIG. 18, the driving axis 230 of the DC motor driving actuator 101 is parallel to that of the motor linkage 175b and the penetrating axis 210, such as when a swash plate is used. In further embodiments, the driving actuator 101 may be coaxial with the motor linkage 175, such as in FIG. 20 where the driving axis 230 of a DC motor driving actuator 101 is coaxial with a barrel cam type motor linkage 175c and FIG. 23 where the driving axis 230 of a piezoelectric actuator 101′ is coaxial with the motor linkage 175d, and the coupler 177 shifts the translational motion to a parallel penetrating axis 210. Accordingly, the driving axis 230 of the driving actuator 101 may be perpendicular, parallel to, or at any oblique angle relative to the penetrating axis 210 of the penetrating member 110. Similarly, the motor linkage 175 may be perpendicular, parallel to, or at any oblique angle relative to the driving axis 230 of the driving actuator 101, which may be defined by a length direction of the extension portion 176 of the motor linkage 175.

Further, the motor linkage 175, and more specifically the extension portion 176, may be at any angle relative to the reservoir axis 220. For instance, the motor linkage 175a and/or extension portion 176a may be parallel to the reservoir axis 220 as shown in FIGS. 15 and 18. In other embodiments, the motor linkage 175a and/or extension portion 176a may be perpendicular to the reservoir axis 220. In still other embodiments, the motor linkage 175a and/or extension portion 176a may be at an oblique angle relative to the reservoir axis 220. Moreover, the penetrating axis 210, reservoir axis 220, driving axis 230, and motor linkage 175 may be at any combination of angles relative to one another, including but not limited to perpendicular, parallel and oblique angles.

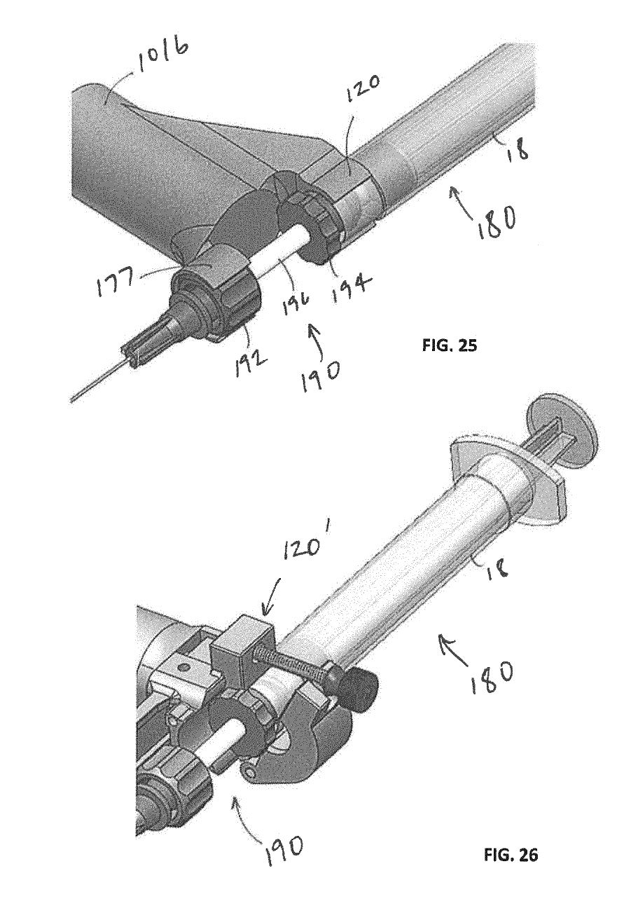

With reference to FIGS. 25 and 26, the device 100 may also include a coupling bracket 120 that selectively attaches the driving actuator 101 to a reservoir 180, such as a syringe body 18 or collection tube. The coupling bracket 120 may be integrally formed with the handpiece 101b, or may be securely attached to the handpiece 101b. The coupling bracket 120 may be of any construction that permits selective attachment and removal of the driving actuator 101 (and device 100) to a reservoir 180, such as by receiving and restraining at least a portion of the reservoir 180. For instance, in one embodiment the coupling bracket 120 may be a snap-fit clip as in FIG. 25 that snaps onto the reservoir 180, such as at one end of a syringe body 18. In other embodiments, as in FIG. 26, the coupling bracket 120′ may include a thumbscrew, threaded rod and hinge clip where the hinge clip is positioned on either side of the reservoir 180 or syringe body 18 and the thumbscrew or threaded rod is engaged to tighten or loosen the hinge clip to secure or release the reservoir 180. These are but a few non-limiting examples, and other mechanical means for selectively adjusting the connection are also contemplated.

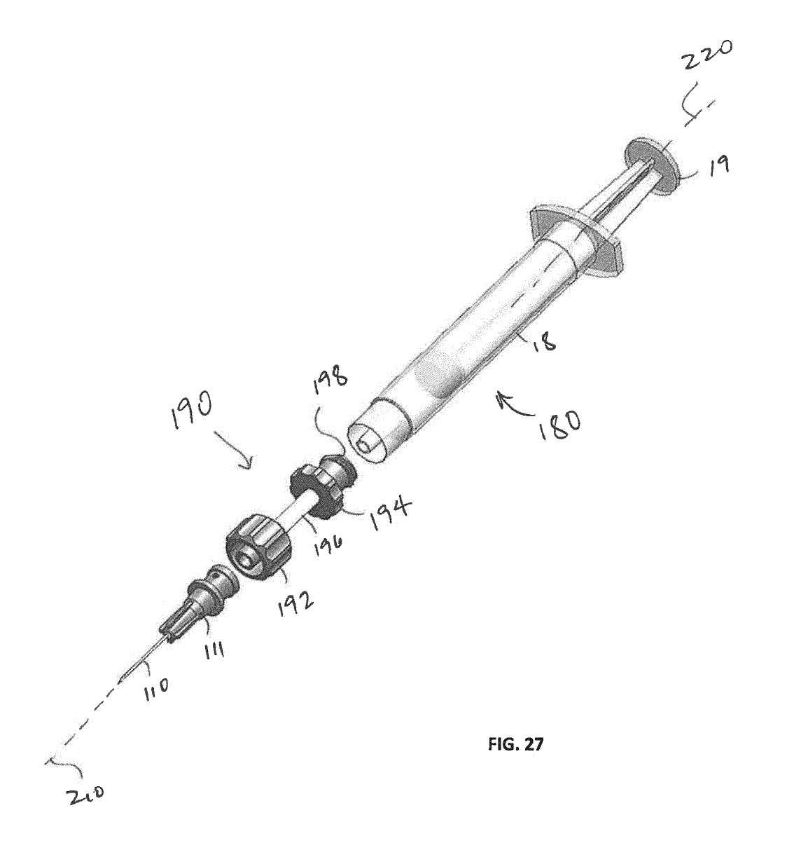

As shown in FIGS. 27-29, in some embodiments the device 100 further includes a hollow member 190 interposed between the penetrating member 110 and the reservoir 180 that it isolates the vibrations from the reciprocating penetrating member 110 so that they are not transferred to the reservoir 180. In other words, the hollow member 190 decouples the vibrations or oscillations from the penetrating member 110 and the reservoir 180 so that the penetrating member 110 reciprocates but the reservoir 180 does not.

The hollow member 190 includes a first end 192 that is attachable in fluid communication to the penetrating member 110, either directly or indirectly through connection to the hub 111. In at least one embodiment, the first end 192 is selectively attachable to one of the penetrating member 110 or hub 111, for connection and removal when desired. In other embodiments, the first end 192 may be integrally formed with either the proximal end of the penetrating member 110 or the hub 111.

In at least one embodiment, the motor linkage 175 discussed previously may connect to the penetrating member 110 or hub 111 through the first end 192 of the hollow member 190. For instance, the motor linkage 175 may be selectively attachable to one or both the hub 111 or first end 192 of the hollow member 190. In the embodiment shown in FIG. 17, the motor linkage 175 engages the first end 192 of the hollow member 190. Specifically, the coupler 177a of the motor linkage 175a may dip onto the first end 192 of the hollow member 190, such as with a snap-fit engagement or other components enabling selective attachment and removal. In other embodiments, as in FIG. 23, the motor linkage 175, 175d may connect to the first end 192 of the hollow member 190 which includes a groove, where a portion of the motor linkage 175d engages the groove to facilitate retention on the first end 192.

The hollow member 190 also includes a second end 194 opposite from the first end 192. The second end 194 has a port 198 that is configured to be attachable in fluid communication with the reservoir 180, such as a syringe body 18. In at least one embodiment, the second end 194 is selectively attachable to a reservoir 180, such as through the port 198, for connection to and switching between syringes. This may be particularly useful when blood samples are collected from multiple specimens/animals, such as in a laboratory environment, or when administering multiple vaccines or medications to a patient.

The first and second ends 192, 194 may be made of any rigid and/or durable material and be of any configuration that will facilitate connection to the penetrating member 110 and reservoir 180 to provide a fluid connection therebetween. The first and second ends 192, 194 may further be configured to provide selective attachment to the penetrating member 110 and reservoir 180 while still providing a fluid tight seal. For instance, in at least one embodiment the first and second ends 192, 194 may be of a Luer type construction, such as a Luer lock or Luer slip, and may be either male or female type connections as would interface with the respective penetrating member 110 or hub 111, or reservoir 180. Accordingly, the first and second ends 192, 194 may provide a quick connect and release to the penetrating member 110 and reservoir 180, respectively.

Further, the first end 192 reciprocates with the penetrating member 110 along the penetrating axis 210 when the driving actuator 101 is activated. The second end 194 remains stationary when the driving actuator 101 is activated, and does not reciprocate with the penetrating member 110. Accordingly, the oscillations or vibrations are isolated between the first and second ends 192, 194.

The hollow member 190 further includes compliant tubing 196 extending between the first and second ends 192, 194. The compliant tubing 196 is constructed and configured to isolate the vibrations and oscillations of the penetrating member 110 so they are not conveyed to the second end 194 of the hollow member 190 or the reservoir 180. For example, in some embodiments the compliant tubing 196 may be as described above regarding the compliant tubing 17 in FIG. 4, where the compliant tubing 17 is sufficiently flexible to permit reciprocating motion of a penetrating member 10 when inline or coaxial with a driving actuator 1 but offset from the syringe 18. The syringe body 18 which is connected to the penetrating member 10 through the compliant tubing 17 is not affected by the oscillations of the penetrating member 10, since the syringe axis is parallel to that of the penetrating member 10. The compliant tubing 17 may be quite flexible in such embodiments to permit sufficient displacement by the penetrating member 10 and still maintain connection to both the penetrating member 10 (or hub 11) and the syringe body 18.

In other embodiments, as in FIGS. 14 and 26-29, the compliant tubing 196 may be axially aligned (coaxial) with both the penetrating axis 210 and reservoir axis 220. In such embodiments, the compliant tubing 196 may be made of a material that exhibits both flexible and stiff physical properties to allow the vibrations or oscillations from the penetrating member 110 to be absorbed by the compliant tubing 196. When receiving vibrations or oscillations from the penetrating member 110, the portion of the compliant tubing 196 at the first end 192 nearest to the penetrating member 110 may partially collapse in the axial direction and expand in the circumferential direction. This absorbs the vibrations so they are not transferred through to the second end 194 of the hollow member 190.

Many factors may contribute to the vibration isolating property of the compliant tubing 196. For instance, the compliant tubing 196 may be sufficiently flexible to absorb (rather than transfer) the vibrations or oscillations from the penetrating member 110 but is also sufficiently stiff to prevent ballooning out in the direction perpendicular to the penetrating axis 210. Accordingly, the compliant tubing 196 may be softer or more flexible in an axial direction but stiffer in the circumferential direction. Some non-limiting examples of materials include silicone and polyurethane, though other materials with similar properties are also contemplated. The compliant tubing 196 may thus have a durometer in the range of 30 A to 70 A, and preferably 50 A. The thickness of the compliant tubing 196 may also contribute to the resilient properties of the compliant tubing 196 that permits vibration absorption and stiffness. For instance, the compliant tubing 196 may have a wall thickness in the range of 0.03 inches to 0.09 inches.

Decoupling the vibration of the penetrating member 110 from the reservoir 180 may be preferable or even required depending on the clinical application. For instance, when collecting blood in the reservoir 180, vibrations to the reservoir 180 could damage the collected blood cells and render any subsequent tests on the samples unusable or unreliable. Further, any reservoir 180 and contents would continually change the resonance frequency of the device 100, which is considered an entire system having the same resonance frequency if the reservoir 180 and its contents are mechanically linked to the driving actuator 101 and penetrating member 110. By decoupling the reservoir 180 and its contents from the penetrating member 110, the mass of the system will not change and the resonance frequency will remain more constant. It will therefore be easier to keep the driving actuator 101 and penetrating member 110 operating at an optimal frequency, or to maintain resonance frequency if drifting occurs since the drifts are likely to have less magnitude. In addition, if the reservoir 180 were part of the system being vibrated, a larger driving actuator 101 capable of more torque would be needed to achieve the same level of reduction of force by the penetrating member 110. By decoupling the reservoir 180 from the penetrating member 110, a smaller, more compact and efficient driving actuator 101 can be used, which also enables the device 100 to be handheld.

As can be seen best from FIG. 29, the hollow member 190 is hollow throughout. The first end 192, compliant tubing 196 and second end 194 each have an inner diameter that is sufficiently large to avoid damaging samples being collected such as red blood cells through turbulence that could lyse or shear the cells, and is sufficiently large to permit passage of fluids that may the thicker such as vaccines or medications in suspension. In at least one embodiment, the inner diameter of the first end 192, compliant tubing 196 and second end 194 are the same as one another. However, in other embodiments, the inner diameters may be different from one another, provided that a fluid tight communication is maintained between each. The inner diameter of the first end 192 may be the same or substantially the same as the diameter of the lumen of the penetrating member 110 such that the hollow member 190 provides fluid communication with the lumen or interior of the penetrating member 110 for fluid collection and delivery through the penetrating member 110. Similarly, the port 198 at the second end 194 of the hollow member 190 may be of the same diameter as a connection point for the reservoir 180, such as the Luer connection on a syringe body 18. Therefore, the hollow member 190 establishes fluid communication between the lumen of the penetrating member 110 and the port 198, and therefore also to a reservoir 180 when connected to the port 198. Notably, this fluid communication between the lumen of the penetrating member 110 and the port 198 remains consistent and uninterrupted regardless of any flexional and tensile deformation the compliant tubing 196 may experience while receiving and absorbing vibrations from the penetrating member 110.

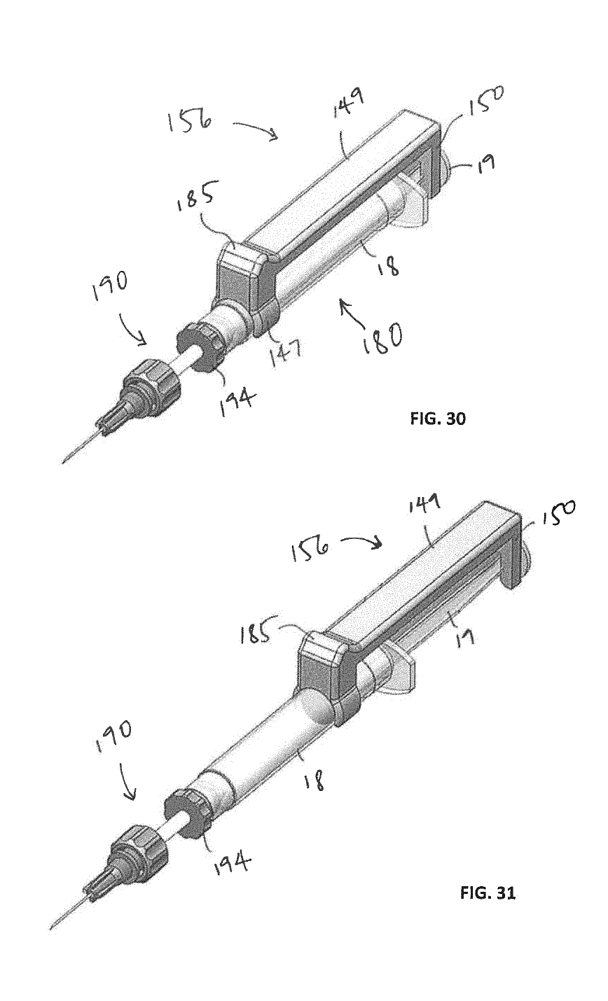

In certain embodiments such as shown in FIGS. 14 and 30-32, the device 100 may also include a slider device 156 that is configured to move one portion of the reservoir 180 relative to another portion of the reservoir 180 for delivery of fluids from the reservoir 180 or collection of fluids into the reservoir 180 through the device 100. For instance, when the reservoir 180 is a syringe 18 as shown in the Figures, the slider device 156 may be used to withdraw and depress a plunger 19 that is slidably inserted and retained within the syringe body 18 for extraction and delivery of fluids, respectively. FIG. 30 shows the slider device 156 in a forward or distal position relative to the syringe body 18. FIG. 31 shows the same slider device 156 in a retracted position where the slider device 156 has been moved in a proximal direction relative to the syringe body 18. The slider device 156 may be similar to the slider device 56 of FIGS. 7A-8D, though it may also be different therefrom in certain embodiments.

With respect to FIGS. 30-32, the slider device 156 includes a guide shaft 149 much like that described above with respect to FIGS. 7A-8D. The guide shaft 49 is a rigid, preferably elongate member that is positionable and movable parallel to the reservoir axis 220. A guide shaft coupling 150 selectively and removably attaches to one portion of the reservoir 180, such as the plunger 19 of a syringe 18, which may be by a snap-fit or other type connection suitable for ready attachment and detachment. An adapter 147 located at another position along the guide shaft 149 is configured to be removably and slidably attachable to the reservoir 180, such as to the exterior surface of the syringe body 18, which also may be by snap-fit or other type connection suitable for ready attachment and detachment. In some embodiments, the guide shaft coupling 150 and adapter 147 may be located at opposite ends of the guide shaft 149. In certain embodiments, such as in FIGS. 30-31, the guide shaft coupling 150 may be dimensioned for gripping the plunger 19 for the application of force to the plunger 19 in order to move, whereas the adapter 147 may be dimensioned to better accommodate a sliding action along the syringe body 18.

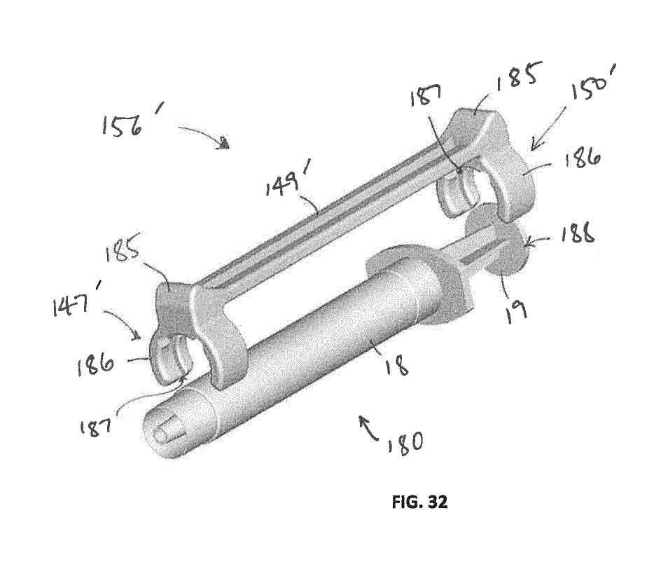

In other embodiments, as in FIG. 32, the guide shaft coupling 150′ and adapter 147′ may have the same geometries and even dimensions such that each can be used interchangeably to connect to either the syringe body 18 or plunger 19. In such embodiments, the sliding device 156′ can be quickly attached to and operated with a reservoir 180 without concern for orientation of the slider device 156′ relative to the reservoir 180. For instance, each of the guide shaft coupling 150′ and adapter 147′ may include a terminal portion 186 configured to at least partially circumferentially engage either the syringe body 18 or flange 188 of a plunger 19, such as by snap fit. This terminal portion 186 allows for both engagement with the syringe body 18 or flange 188 of the plunger 19, but also permits sliding along the syringe body 18. The terminal portion 186 may further include a groove 187 formed therein that is dimensioned to receive the flange 188 of the plunger 19. The groove 187 increases the retention of the flange 188 of the plunger 19 in the terminal portion 186, thereby increasing the ease with which the slider device 156′ is operated.

The slider device 156 further includes at least one engagement portion 185 that facilitates the application of force to the guide shaft 149. For instance, the engagement portion 185 may be pressed or otherwise engaged by a user of the device 100 and/or slider device 156 to move the slider device 156 axially along the reservoir 180. In certain embodiments, as in FIGS. 30-31, the slider device 156 may include a single engagement portion 185 located anywhere along the guide shaft 149, such as at one end or the other. In other embodiments, as seen in FIG. 32, the slider device 156′ may include a plurality of engagement portions 185, such as one at each end of the guide shaft 149′. The engagement portion 185 may be pressed by the thumb or finger of a user of the device 100 and while pressing, force may be applied with the thumb or finger in a distal or proximal direction. Distally directed force pushes the slider device 156, 156′ in a distal direction toward the penetrating member 110. The connection of the guide shaft coupling 150, 150′ with the remote portion of the reservoir 180 contracts the space within the reservoir 180 and pushes the fluid in the reservoir 180 through the hollow member 190 and penetrating member 110. In embodiments where the reservoir 180 is a syringe 18, distally directed movement of the slider device 156 pushes the plunger 19 into the syringe body 18, as in FIG. 30. In contrast, proximally directed force on the engagement portion 185 moves the slider device 156 away from the penetrating member 110 and into a retracted position, as in FIG. 31. This motion draws fluid through the penetrating member 110 and hollow compliant tubing 190 into the reservoir 180 for collection. The connection of the guide shaft coupling 150, 150′ with the plunger 19 transfers the force applied to the engagement portion 185 to the plunger 19 as well, thus moving the plunger 19. Accordingly, the guide shaft 149, 149′ and plunger 19 move together, the motion of the plunger 19 being driven and controlled by the motion of the guide shaft 149, 149′. Moreover, this motion of the guide shaft 149, 149′ and plunger 19 is independent and separate from the reciprocating motion of the penetrating member 110 and driving actuator 101.

Although described as pressing and/or applying force to the engagement portion 185, it should be appreciated that force or pressure can be applied to any location along the slider device 156, 156′, such as any point along the guide shaft 149, 149′ or even guide shaft coupling 150, 150′, to move the slider device 156, 156′ relative to the reservoir 180. The engagement portion 185 may be raised or elevated above the level of the guide shaft 149, 149′, such as protrusion, lowered from the level of the guide shaft 149, 149′ as in a detent, include frictional elements, or provide other similar structure to increase the ease of applying sliding force to the slider device 156, 156′. The slider device 156, 156′ and the easy to use engagement portion 185, together with the handpiece 101b, enables one-handed operation of both the device 100 for penetration and the slider device 156, 156′ for delivery and/or collection of fluids following penetration. It is much easier for the user to operate and makes the delivery or collection of fluids a less traumatic experience.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed:

1. A device for penetrating tissue, comprising:
a driving actuator having a driving axis and configured to linearly reciprocate a penetrating member;
said penetrating member having a proximal end, an opposite distal end, and a lumen extending along a penetrating axis from said proximal end to said distal end, said penetrating member interconnected to said driving actuator and configured to reciprocate along said penetrating axis; and
a hollow member having a first end in fluid communication with said lumen of said penetrating member, a second end forming a port for selective fluid communication, and compliant tubing between said first and second ends, said hollow member providing consistent fluid communication between said lumen of said penetrating member and said port during reciprocation of said penetrating member.

2. The device of claim 1, wherein said hollow member is selectively attachable to said penetrating member.

3. The device of claim 1, further comprising a hub at said proximal end of said penetrating member, wherein said hollow member is one of (i) selectively attachable to said hub, and (ii) integral with said hub.

4. The device of claim 3, wherein said first end of said hollow member is selectively attachable to said hub.

5. The device of claim 1, wherein said hollow member is axially aligned with said penetrating axis.

6. The device of claim 1, further comprising a fluid reservoir selectively attachable to said port at said second end of said hollow member and in fluid communication therewith.

7. The device of claim 6, wherein said fluid reservoir is a syringe.

8. The device of claim 6, said driving actuator further comprising a handpiece having a coupling bracket that is releasably attachable to said fluid reservoir.

9. The device of claim 8, further comprising a guide shaft removably connectable to a plunger that is slidably insertable in said fluid reservoir, said guide shaft and said plunger being selectively movable together independent from said linear reciprocation of said penetrating member.

10. The device of claim 9, said fluid reservoir further comprising a reservoir axis, wherein said guide shaft is parallel to said reservoir axis.

11. The device of claim 9, said handpiece further comprising an exterior surface having a power button, said guide shaft further comprising an engagement portion configured to receive force for selective movement of said guide shaft, wherein said handpiece is sized and dimensioned to facilitate one-handed operation of said device and said guide shaft.

12. The device of claim 6, said fluid reservoir further comprising a reservoir axis.

13. The device of claim 12, wherein said reservoir axis is one of (i) coaxial with, (ii) parallel to, and (iii) at an oblique angle relative to said penetrating axis.

14. The device of claim 13, wherein said driving axis is one of (I) perpendicular to, (ii) parallel to, and (iii) at an oblique angle relative to said reservoir axis.

15. The device of claim 1, wherein said driving axis is one of (i) perpendicular to, (ii) parallel to, and (iii) at an oblique angle relative to said penetrating axis.

16. The device of claim 1, further comprising a motor linkage interconnecting said driving actuator and said penetrating member, said motor linkage being one of (i) perpendicular to, (ii) parallel to, and (iii) at an oblique angle relative to said driving axis.

17. The device of claim 1, further comprising a hub at said proximal end of said penetrating member; said first end of said compliant member selectively attachable to said hub; and a motor linkage extending from said driving actuator, said motor linkage being selectively connectable to at least one of said hub and said first end of said hollow member.

18. The device of claim 17, wherein said first end of said hollow member includes a groove and said motor linkage engages said groove in selectively connecting to said first end of said hollow member.

19. The device of claim 17, wherein said motor linkage further comprises a coupler that is selectively connectable to at least one of said hub and said first end of said hollow member.

20. The device of claim 1, wherein said driving actuator is one of a voice coil, piezoelectric element, DC motor, and a flextensional transducer.

21. The device of claim 1, further comprising a controller in electrical communication with said driving actuator and configured to operate said driving actuator according to one of:
(i) a preselected operating frequency based on tissue to be penetrated, wherein said preselected operating frequency is sufficient to offset at least a portion of damping of oscillatory displacement amplitude resulting from a resonant frequency shift from air to tissue upon insertion of said penetrating member into tissue, wherein said preselected operating frequency is selected from the group consisting of:
a. the resonance frequency of the penetrating member in tissue;
b. a frequency higher than a resonant frequency of said penetrating member in air;
c. in the range of ⅓ to ½ octave higher than the resonant frequency of said penetrating member in air; and
d. in the range of 95-150 Hz;
(ii) an operating frequency that is variably adjustable during use based on a feedback loop to maintain said operating frequency near a optimal frequency; and
(iii) optimal driving parameters based on the type of said driving actuator, said optimal driving parameters including settings for torque, frequency and voltage.

22. A slider device, comprising:
a guide shaft positionable parallel to a reservoir axis of a reservoir;
a guide shaft coupling extending from said guide shaft and selectively attachable to a first portion of said reservoir;
an adapter extending from said guide shaft and slidably attachable to a second portion of said reservoir, said first and second portions of said reservoir being spaced apart from one another;
wherein said guide shaft and said guide shaft coupling are collectively configured so that application of force to said guide shaft in a proximal or distal direction moves said second portion of said reservoir in the same proximal or distal direction when said guide shaft coupling is attached thereto.

23. The slider device of claim 22, wherein said guide shaft and said guide shaft coupling are rigid.

24. The slider device of claim 22, wherein at least one of said guide shaft coupling and said adapter are integrally formed with said guide shaft.

25. The slider device of claim 22, wherein said guide shaft coupling and said adapter are located at opposite ends of said guide shaft.

26. The slider device of claim 22, wherein said guide shaft coupling and said adapter have the same geometries.

27. The slider device of claim 22, wherein at least one of said guide shaft coupling and said adapter are connectable to said reservoir by snap-fit connection.

28. The slider device of claim 22, wherein said guide shaft is elongate and has a length parallel to said reservoir axis.

29. The slider device of claim 22, wherein said guide shaft is axially movable along said reservoir axis.

30. The slider device of claim 22, wherein said reservoir includes a syringe body and plunger slidably inserted in said syringe body, said guide shaft coupling is selectively attachable to said plunger, said adapter is connectable to said syringe body, and movement of said guide shaft results in axial movement of said plunger into and out of said syringe body.

31. The slider device of claim 30, wherein said guide shaft coupling is selectively attachable to one of a flange and an elongate portion of said plunger.

32. The slider device of claim 30, wherein said adapter is slidably connectable to said syringe body.

33. The slider device of claim 22, further comprising at least one engagement portion on said guide shaft, said at least one engagement portion configured to receive force resulting in motion of said guide shaft.

34. The slider device of claim 33, wherein said engagement portion includes at least one of a protrusion, detent, and frictional element.
